(19) United States
(12) Patent Application Publication
(10) Pub. No.: US 2010/0068775 A1
(43) Pub. Date: Mar. 18, 2010

(54) NOVEL GENES FOR THE FERMENTATIVE PRODUCTION OF HYDROXYTYROSOL

(76) Inventors: Jihane Achkar, Zurich (CH); Abel Ferrandez, Basel (CH); Theodorus Sonke, Guttecoven (NL); Marcel G. Wubbolts, Sittard (NL)

Correspondence Address: NIXON & VANDERHYE, PC, 901 NORTH GLEBE ROAD, 11TH FLOOR, ARLINGTON, VA, 22203, US

(21) Appl. No.: 12/516,415

(22) PCT Filed: Nov. 27, 2007
(86) PCT No.: PCT/EP07/10211

§ 371 Date: Jun. 30, 2009

(30) Foreign Application Priority Data
Nov. 27, 2006 (EP) 06024493.6

(51) Int. Cl. C12P 7/22; C12N 15/63; C12N 9/02; C12N 1/21; C12N 15/70

(52) U.S. Cl. 435/156; 435/320.1; 435/189; 435/252.33; 435/471

(57) ABSTRACT

The present invention relates to the use of polynucleotides and polypeptides as biotechnological tools in the production of hydroxytyrosol from microorganisms, whereby a modification of said polynucleotides and/or encoded polypeptides has a direct or indirect impact on yield, production, and/or efficiency of production of hydroxytyrosol in said microorganism. The invention also features polynucleotides comprising the full length polynucleotide sequences of the novel genes and fragments thereof, the novel polypeptides encoded by the polynucleotides and fragments thereof, as well as their functional equivalents. Also included are methods/processes of using the polynucleotides and modified polynucleotide sequences to transform host microorganisms. The invention also relates to genetically engineered microorganisms and their use for the production of hydroxytyrosol.

The present invention relates to the use of polynucleotides and polypeptides as biotechnological tools in the production of hydroxytyrosol from microorganisms, whereby a modification of said polynucleotides and/or encoded polypeptides has a direct or indirect impact on yield, production, and/or efficiency of production of the fermentation product in said microorganism. The invention also features polynucleotides comprising the full length polynucleotide sequences of the novel genes and fragments thereof, the novel polypeptides encoded by the polynucleotides and fragments thereof, as well as their functional equivalents. Also included are methods/processes of using the polynucleotides and modified polynucleotide sequences to transform host microorganisms. The invention also relates to genetically engineered microorganisms and their use for the production of hydroxytyrosol.

Hydroxytyrosol (hereafter called Hy-T) is a potent antioxidant found in olives, thus present in high abundance in olive mill waste waters. Hy-T has been associated with the lower mortality and incidence of cancer in Mediterranean regions and has been attributed cardio-protective properties. There has been therefore an increased interest in the manufacturing and commercialization of Hy-T as nutritional supplement.

Currently, hydroxytyrosol is commercially available only in the form of enriched olive extracts.

Methods for the chemical synthesis of Hy-T have been described, but they make use of environmentally hazardous products such as organic solvents, strong acids, hydrides and/or cyanides. Therefore, over the past years, other approaches to manufacture Hy-T using different extraction methods and/or microbial conversions, which would be more economical as well as ecological, have been investigated.

For example, EP-A-1,623,960 teaches on the recovery of a structural analogue of Hy-T such as tyrosol from olive mill wastewaters via expensive procedures such as microfiltration, ultrafiltration, nanofiltration and reverse osmosis followed by oxidation with heavy metal based catalysts. Further Bouzid O., et al. (Proc. Biochem. (2005) 40: 1855-1862) discloses a method to enrich oil by-products in Hy-T by their treatment with cells of Aspergillus niger enriched in cinnamoyl esterases. Several other examples for the extraction of Hy-T from olive oil, olive tree leaves or olive oil production waste waters can be found, these procedures being developed at low yields, requiring expensive extraction processes and the use of toxic compounds such as organic solvents, or hazardous strong acid treatments.

Further, WO/02/16628 discloses a method for the transformation of tyrosol in vitro making use of purified mushroom tyrosinase. This enzymatic procedure has as main disadvantages the elevated cost of a purified enzyme, as well as the intrinsic instability of enzymes isolated from their natural cellular environment. Furthermore, reaction conditions in this method are restricted to phosphate solutions buffered at pH 7, and the use of room temperature, making use of costly protein removing systems such as molecular size discriminating membranes and purification methods based on techniques such as high performance liquid chromatography (HPLC) of high cost for industrial application purposes. It is therefore desirable to make use of technologies offering a broader range of reaction conditions for their applicability and not restricting themselves to the use of purified mushroom tyrosinase. No enzyme other than mushroom tyrosinase is found in the prior art capable of transforming organic compounds such as, for example, tyrosol to Hy-T.

Finally, the ability to transform the precursor tyrosol to hydroxytyrosol has been reported in a few microorganisms, but there is no previous report indicating how to increase the ability of microorganisms to transform organic compounds such as, for example, tyrosol to Hy-T. Furthermore, one of the main disadvantages of the approaches cited above is the use of undesirable human opportunistic pathogens such as Pseudomonas aeruginosa (Allouche N., et al. Appl. Environ. Microbiol. (2004) 70: 2105-2109) or Serratia marcensces (Allouche N., et al. J. Agric. Food Chem. (2005) 53: 6525-6530). Furthermore, these organisms are described as not only capable of transforming tyrosol to Hy-T, but also of utilizing the costly and highly valuable substrate tyrosol as carbon source i.e. of eliminating the substrate and its product Hy-T from the culture medium. Although prior art teaches how to transform tyrosol (2-(4-hydroxyphenyl)ethanol) to Hy-T, surprisingly there is no known biotechnological method described so far for the transformation of organic compounds other than tyrosol to Hy-T.

Consequently, there is a need to develop optimized fermentation systems for the microbial production of Hy-T either for the transformation of a broader range of organic compounds or to get higher yields than with the systems described above in order to produce Hy-T making use of renewable resources.

It has now been found that two groups of enzymes involved in the metabolism of aromatic compounds play an important role in the biotechnological production of Hy-T. It has also been found, that by using polynucleotide sequences encoding these enzymes in a microorganism, such as for example Escherichia coli, the fermentation for Hy-T by said microorganism can be even greatly improved.

More precisely, it has been found that the enzymes capable of improving fermentative production of Hy-T are involved either in the elaboration of the Hy-T specific aromatic ring hydroxylation pattern (HP enzymes) or in the elaboration of the correct functional group of the Hy-T side chain (FG enzymes). Polynucleotides according to the invention and proteins encoded by these polynucleotides are herein abbreviated by HP and FG.

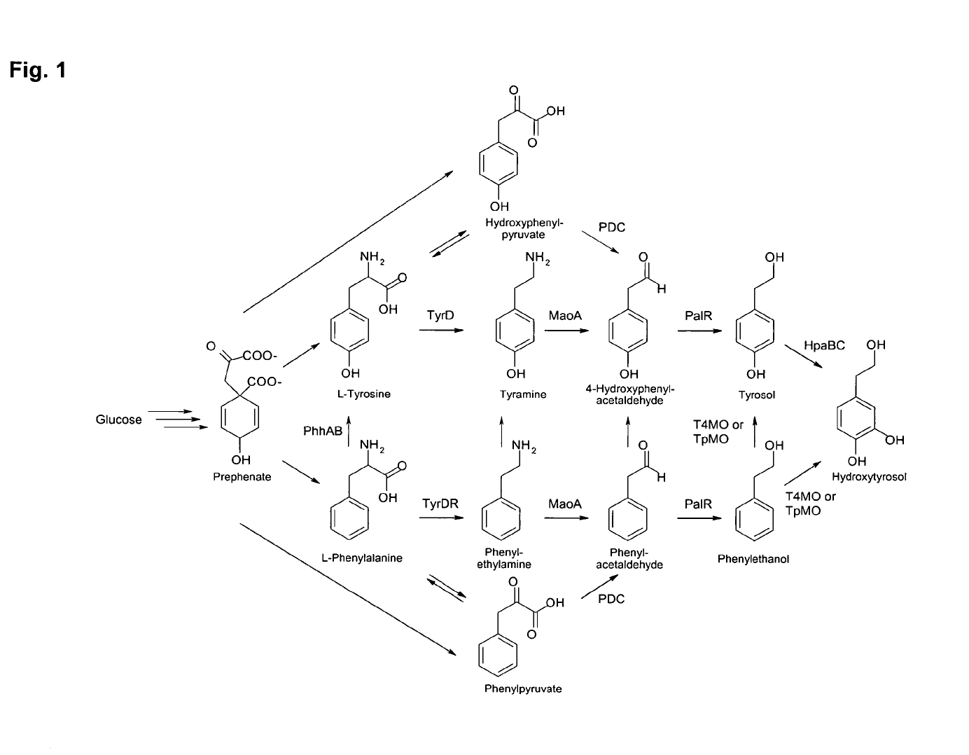

The enzymes involved in the biosynthesis of hydroxytyrosol and which are capable of improving Hy-T production are shown in FIG. 1.

HP and FG encoding polynucleotides are known in the art. The candidates which are able to improve fermentative production of Hy-T according to the present invention are selected from the group consisting of:
1. Polynucleoteides encoding enzymes capable of transforming tyrosol into Hy-T and/or L-tyrosine into L-3,4-dihydroxyphenylalanine comprising the polynucleotide sequence according to SEQ ID NO:1; SEQ ID NO:38 and SEQ ID NO:40 or variants thereof SEQ ID NO:1 corresponds to a tyrosinase from Pycnoporus sanguineus, a HP enzyme according to SEQ ID NO:2. SEQ ID NO:38 and SEQ ID NO 40 correspond to two tyrosinases from Agaricus bisporus, HP enzymes according to SEQ ID NO:39 and SEQ ID NO: 41.
2. Polynucleotides encoding enzymes capable of transforming phenylacetaldehyde to phenylethanol and/or 4-hydroxyphenylacetaldehyde to tyrosol comprising the polynucleotide sequence according to SEQ ID NO:3 or variants thereof. SEQ ID NO:3 corresponds to the gene palR gene from Rhodococcus erythropolis which encodes a phenylacetaldehyde reductase (PalR), a FG-enzyme according to SEQ ID NO:4, that catalyzes the asymmetric reduction of aldehydes or ketones to chiral alcohols. This NADH-dependent enzyme belongs to the family of zinc-containing medium-chain alcohol dehydrogenases.
3. Polynucleotides encoding enzymes capable of transforming tyrosol to Hy-T comprising the polynucleotide sequence according to SEQ ID NO:5 and/or SEQ ID NO:7 or variants thereof.
   The hpaB and hpaC genes from Escherichia coli W which correspond to SEQ ID NO:5 and SEQ ID NO:7 respectively express a two-components enzyme, 4-hydroxyphenylacetate 3-monooxygenase. The HP-enzyme (HpaBC) was reported to be a two-component flavin-dependent monooxygenase that catalyzes the hydroxylation of 4-hydroxyphenylacetate into 3,4-dihydroxyphenylacetate. The large component (HpaB; protein SEQ ID NO:6,) is a reduced flavin-utilizing monooxygenase. The small component (HpaC, protein SEQ ID NO:8) is an oxido-reductase that catalyzes flavin reduction using NAD(P)H as a reducent.
4. Polynucleotides encoding enzymes capable of transforming L-phenylalanine to 2-phenylethylamine and/or L-tyrosine to tyramine comprising the polynucleotide sequence according to SEQ ID NO:9 or variants thereof.
   SEQ ID NO:9 corresponds to the gene tyrDR from Pseudomonas putida which encodes an FG-enzyme (TyrDR) belonging to the enzymatic family of aromatic-L-amino-acid decarboxylases, such as, for example, L-phenylalanine and L-tyrosine decarboxylases according to SEQ ID NO:10.
5. Polynucleotides encoding enzymes capable of transforming 2-phenylethylamine to phenylacetaldehyde and/or tyramine to 4-hydroxyphenylacetaldehyde comprising the polynucleotide sequence according to SEQ ID NO:11 or variants thereof SEQ ID NO:11 corresponds to the maoA gene from E. coli K-12 which encodes a monoamine oxidase (MaoA), a copper-containing FG-enzyme according to SEQ ID NO:12 using 3,4,6-trihydroxyphenylalanine quinone as cofactor that catalyzes the oxidative deamination of monoamines to produce the corresponding aldehyde. Oxygen is used as co-substrate with the amine, and ammonia and hydrogen peroxide are by-products of the reaction in addition to the aldehyde.
6. Polynucleotides encoding enzymes capable of transforming L-tyrosine to tyramine comprising the polynucleotide sequence according to SEQ ID NO:13 or variants thereof.
   SEQ ID NO:13 corresponds to the tyrD gene which encodes a tyrosine decarboxylase (TyrD) from Methanocaldococcus jannaschii according to SEQ ID NO:14, a lyase which is an FG-enzyme that catalyzes the removal of the carboxylate group from the amino acid tyrosine to produce the corresponding amine tyramine and carbon dioxide using pyridoxal 5′-phosphate as a necessary cofactor.
7. Polynucleotides encoding enzymes capable of transforming phenylpyruvate to phenylacetaldehyde and/or hydroxyphenylpyruvate to 4-hydroxyphenylactealdehyde comprising the polynucleotide sequence according to SEQ ID NO:16 or variants thereof. SEQ ID NO:16 corresponds to the PDC gene from Acinetobacter calcoaceticus which encodes an FG-enzyme (SEQ ID NO:17) that has the activity of a phenylpyruvate decarboxylase.
8. Polynucleotides encoding hydroxylating enzymes such as toluene monooxygenases which are capable of transforming phenylethanol to tyrosol and/or Hy-T. For example, toluene para-monooxygenase (TpMO) from Ralstonia pickettii PKO1 and toluene 4-monooxygenase (T4MO) from Pseudomonas mendocina KR1. Both enzymes are multi-component non-heme diiron monooxygenases encoded by six genes and comprising a hydroxylase component structured in three alpha-, beta-, and gamma-subunits that assemble into an HP-enzyme.
   SEQ ID NO:18, 20 and 22 encode the alpha, beta and gamma subunits of TpMO, respectively, and SEQ ID NO: 19, 21 and 23 represent the protein sequences of these subunits, respectively.
   SEQ ID NO:24, 26 and 28 encode the alpha, beta and gamma subunits of T4MO, respectively, and SEQ ID NO 25, 27 and 29 represent the protein sequences of these subunits, respectively.
9. Polynucleotides encoding enzymes capable of transforming L-phenylalanine to L-tyrosine comprising the polynucleotide sequences according to
   SEQ ID NO:30 and/or SEQ ID NO:32; or
   SEQ ID NO:34 and/or SEQ ID NO:36
   or variants thereof.
   These two pairs of sequences correspond to the phhAB genes which encode a two-component hydroxylase (HP-enzyme). The large component (PhhA) is represented by SEQ ID NO:30 and SEQ ID NO:34 encoding the proteins according to SEQ ID NO:31 and SEQ ID NO:35, respectively, which are phenylalanine-4-hydroxylase enzymes from P. aeruginosa and P. putida, respectively. The small component (PhhB) is represented by SEQ ID NO:32 and SEQ ID NO:36 encoding the proteins according to SEQ ID NO:33 and SEQ ID NO 37, respectively, which are pterin-4-alpha-carbinolamine dehydratase enzymes from P. aeruginosa and P. putida, respectively.

It is one object of the present invention to provide the use of a polynucleotide as defined above in the biotechnological production of Hy-T.

Furthermore, it is also an object of the present invention to provide a process for producing a host cell which is genetically engineered, for example transformed by such polynucleotide (DNA) sequences or vectors comprising polynucleotides as defined above. This may be accomplished, for example, by transferring polynucleotides as exemplified herein into a recombinant or non-recombinant host cell that may or may not contain an endogenous equivalent of the corresponding gene.

Such a transformed cell is also an object of the invention, wherein the activity of the enzyme expressed by the transfected polynucleotide is enhanced so that the yield of Hy-T is increased.

If the host cell of choice is not capable of producing L-phenylalanine, and/or L-tyrosine, and/or prephenate, such host cells can be altered to produce Hy-T by supplying either of these compounds or mixtures thereof to the reaction medium.

Finally, it is also an object of the present invention to provide a process for the direct fermentative production of Hy-T by using a genetically engineered host cell as defined above.

Advantageous embodiments of the invention become evident from the dependent claims. These and other aspects and embodiments of the present invention should be apparent to those skilled in the art from the teachings herein.

The term “direct fermentation”, “direct production”, “direct conversion”, “direct bioconversion”, “direct biotransformation” and the like is intended to mean that a microorganism is capable of the conversion of a certain substrate into the specified product by means of one or more biological conversion steps, without the need of any additional chemical conversion step. A single microorganism capable of directly fermenting Hy-T is preferred.

As used herein, “improved” or “improved yield of Hy-T” or “higher yield” or “improved bioconversion ratio” or “higher bioconversion ratio” caused by a genetic alteration means an increase of at least 5%, 10%, 25%, 30%, 40%, 50%, 75%, 100%, 200% or even more than 500%, compared to a cell which is not genetically altered. Such unaltered cells are also often referred to as wild type cells.

The term “genetically altered” or “genetically engineered” means any mean of changing the genetic material of a living organism. It can involve the production and use of recombinant DNA, but other methods are available and are known to those skilled in the art to produce genetically altered microorganisms such as, for example, but not limited to, chemical treatments or exposure to ultraviolet or X-Ray irradiation. More in particular it is used to delineate the genetically engineered or modified organism from the naturally occurring organism. Genetic engineering may be done by a number of techniques known in the art, such as e.g. gene replacement, gene amplification, gene disruption, transfection, transformation using plasmids, viruses, or other vectors. A genetically modified organism, e.g. genetically modified microorganism, is also often referred to as a recombinant organism, e.g. recombinant microorganism.

In a preferred embodiment a polynucleotide encoding a protein selected from the group defined above, is transferred into a recombinant or non-recombinant microorganism—hereinafter also called host cell—in such a way that it leads to an improved yield and/or efficiency of production of Hy-T produced by the host cell compared to the wild type counterpart of said cell.

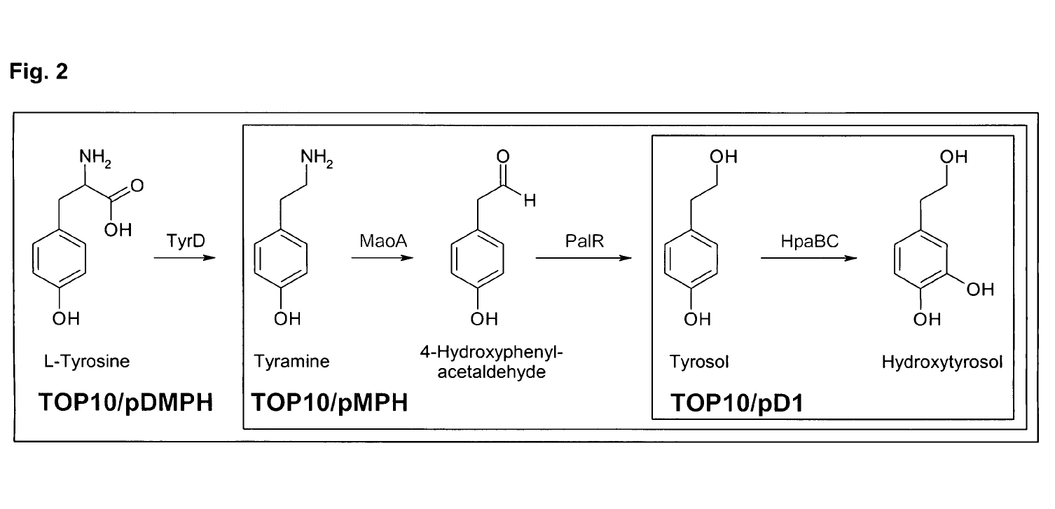

In an other embodiment at least two, preferably at least three or four or five polynucleotides encoding a protein selected from the group defined above, are transferred into a recombinant or non-recombinant microorganism—hereinafter also called host cell—in such a way that it leads to an improved yield and/or efficiency of production of Hy-T produced by the host cell compared to the wild type counterpart of said cell. Preferred polynucleotides for such combinations are hpaBC, maoA, palR, and tyrD. The enzyme reactions carried out by the corresponding polypeptides HpaBC, MaoA, PalR, and TyrD are described in FIG. 2.

Any cell that serves as recipient of the foreign nucleotide acid molecules may be used as a host cell, such as for instance a cell carrying a replicable expression vector or cloning vector or a cell being genetically engineered or genetically altered by well known techniques to contain desired gene(s) on its chromosome(s) or genome. The host cell may be of prokaryotic or eukaryotic origin, such as, for instance bacterial cells, animal cells, including human cells, fungal cells, including yeast cells, and plant cells. Preferably the host cell is a microorganism. More preferably the microorganism belongs to bacteria. The term bacteria includes both Gram-negative and Gram-positive microorganisms. Examples of Gram-negative bacteria are, for example, any from the genera Escherichia, Gluconobacter, Rhodobacter, Pseudomonas, and Paracoccus. Gram-positive bacteria are selected from, but not limited to any of the families Bacillaceae, Brevibacteriaceae, Corynebacteriaceae, Lactobacillaceae, and Streptococcaceae and belong especially to the genera Bacillus, Brevibacterium, Corynebacterium, Lactobacillus, Lactococcus and Streptomyces. Among the genus Bacillus, B. subtilis, B. amyloliquefaciens, B. licheniformis and B. pumilus are preferred microorganisms in the context of the present invention. Among Gluconobacter, Rhodobacter and Paracoccus genera G. oxydans, R. sphaeroides and P. zeaxanthinifaciens are preferred, respectively.

Examples of yeasts are Saccharomyces, particularly S. cerevisiae. Examples of other preferred fungi are Aspergillus niger and Penicillium chrysogenum.

Microorganisms which can be used in the present invention in order to improve the direct production of Hy-T may be publicly available from different sources, e.g., Deutsche Sammlung von Mikroorganismen and Zellkulturen (DSMZ), Mascheroder Weg 1B, D-38124 Braunschweig, Germany, American Type Culture Collection (ATCC), P.O. Box 1549, Manassas, Va. 20108 USA or Culture Collection Division, NITE Biological Resource Center, 2-5-8, Kazusakamatari, Kisarazu-shi, Chiba, 292-0818, Japan (formerly: Institute for Fermentation, Osaka (IFO), 17-85, Juso-honmachi 2-chome, Yodogawa-ku, Osaka 532-8686, Japan).

In a preferred embodiment of the invention, the host cell is a non-pathogenic microorganism.

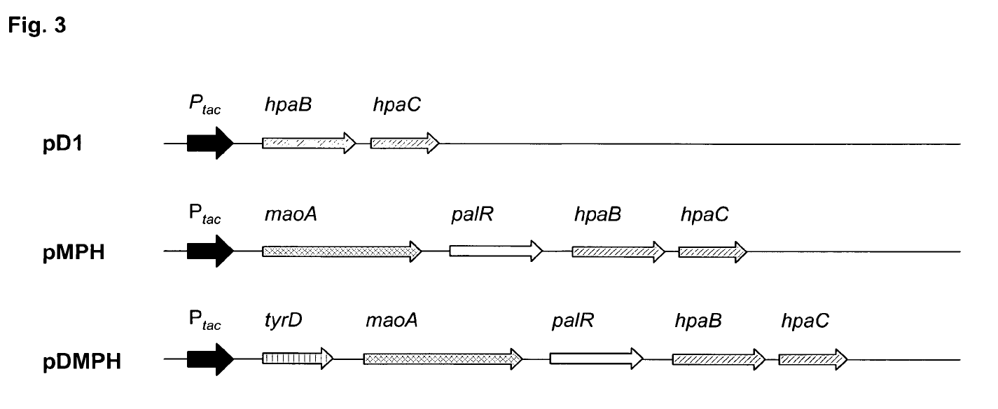

Preferred examples of microorganisms according to the invention derive from the Escherichia coli K-12 strain TOP10, which is available from Invitrogen, and comprise plasmids as shown in FIG. 3.

In FIG. 3 all genes were inserted in the multiple cloning site (MCS) of cloning vector pJF119EH (Furste, J. P. et al., Gene (1986) 48: 119-131) which also carries the ampicillin resistance gene (bla): tyrD, L-tyrosine decarboxylase from Methanocaldococcus jannaschii; maoA, monoamine oxidase from E. coli MG1655; palR, phenylacetaldehyde reductase from Rhodococcus erythropolis (DSM 43297); HpaBC, 4-hydroxyphenylacetic acid 3-monooxygenase operon from E. coli W (ATCC 11105).

In particular, the present invention is related to a process for the direct production of Hy-T wherein at least one—preferably a combination—of polynucleotides or modified polynucleotides disclosed herein are introduced into a suitable microorganism, the recombinant microorganism is cultured under conditions that allow the production of Hy-T in high productivity, yield, and/or efficiency, the produced fermentation product is isolated from the culture medium and optionally further purified.

Several enzyme substrates may be used as starting material in the above-mentioned process. Compounds particularly suited as starting material are prephenate, L-tyrosine, L-phenylalanine, L-3,4-dihydroxyphenylalanine, 4-hydroxyphenylpyruvate, tyramine, 2-phenylethylamine, dopamine, phenylpyruvate, 4-hydroxyphenylacetaldehyde, phenylacetaldehyde, tyrosol, 2-(3-hydroxyphenyl)ethanol, phenylethanol or mixtures thereof.

Conversion of the substrate into Hy-T in connection with the above process using a microorganism means that the conversion of the substrate resulting in Hy-T is performed by the microorganism, i.e. the substrate may be directly converted into Hy-T. Said microorganism is cultured under conditions which allow such conversion from the substrate as defined above.

A medium as used herein for the above process using a microorganism may be any suitable medium for the production of Hy-T. Typically, the medium is an aqueous medium comprising for instance salts, substrate(s), and a certain pH. The medium in which the substrate is converted into Hy-T is also referred to as the production medium.

“Fermentation” or “production” or “fermentation process” or “biotransformation” or “bioconversion” or “conversion” as used herein may be the use of growing cells using any cultivation medium, conditions and procedures known to the skilled person, or the use of non-growing so-called resting cells, after they have been cultivated by using any growth medium, conditions and procedures known to the skilled person, under appropriate conditions for the conversion of suitable substrates into desired products such as Hy-T.

As used herein, resting cells refer to cells of a microorganism which are for instance viable but not actively growing due to omission of an essential nutrient from the medium, or which are growing at low specific growth rates [μ], for instance, growth rates that are lower than 0.02 h⁻¹, preferably lower than 0.01 h⁻¹. Cells which show the above growth rates are said to be in a “resting cell mode”. Microorganisms in resting cell mode may be used as cell suspensions in a liquid medium, be it aqueous, organic, or a mixture of aqueous and organic solvents; or as flocculated or immobilized cells on a solid phase, be it a porous or polymeric matrix.

The process of the present invention may be performed in different steps or phases. In one step, referred to as step (a) or growth phase, the microorganism can be cultured under conditions that enable its growth. In another step, also referred to as step (b) or transition phase, cultivation conditions can be modified so that the growth rate of the microorganism decreases until a resting cell mode is reached. In yet another step, also referred to as step (c) or production phase, Hy-T is produced from a substrate in the presence of the microorganism. In processes using resting cells, step (a) is typically followed by steps (b) and (c). In processes using growing cells, step (a) is typically followed by step (c).

Growth and production phases as performed in the above process using a microorganism may be performed in the same vessel, i.e., only one vessel, or in two or more different vessels, with an optional cell separation step between the two phases. The produced Hy-T can be recovered from the cells by any suitable means. Recovery means for instance that the produced Hy-T may be separated from the production medium. Optionally, the thus produced Hy-T may be further processed.

For the purpose of the present invention relating to the above process, the terms “growth phase”, “growing step”, “growth step” and “growth period” are used interchangeably herein. The same applies for the terms “production phase”, “production step”, “production period”.

One way of performing the above process may be a process wherein the microorganism is grown in a first vessel, the so-called growth vessel, as a source for the resting cells, and at least part of the cells are transferred to a second vessel, the so-called production vessel. The conditions in the production vessel may be such that the cells transferred from the growth vessel become resting cells as defined above. Hy-T is produced in the second vessel and recovered therefrom.

In connection with the above process, the growing step can be performed in an aqueous medium, i.e. the growth medium, supplemented with appropriate nutrients for growth under aerobic conditions. The cultivation may be conducted, for instance, in batch, fed-batch, semi-continuous or continuous mode. The cultivation period may vary depending on the kind of cells, pH, temperature and nutrient medium to be used, and may be for instance about 10 h to about 10 days, preferably about 1 to about 10 days, more preferably about 1 to about 5 days when run in batch or fed-batch mode, depending on the microorganism. If the cells are grown in continuous mode, the residence time may be for instance from about 2 to about 100 h, preferably from about 2 to about 50 h, depending on the microorganism. If the microorganism is selected from bacteria, the cultivation may be conducted for instance at a pH of about 3.0 to about 9.0, preferably about 4.0 to about 9.0, more preferably about 4.0 to about 8.0, even more preferably about 5.0 to about 8.0. If algae or yeast are used, the cultivation may be conducted, for instance, at a pH below about 7.0, preferably below about 6.0, more preferably below about 5.5, and most preferably below about 5.0. A suitable temperature range for carrying out the cultivation using bacteria may be for instance from about 13° C. to about 40° C., preferably from about 18° C. to about 37° C., more preferably from about 13° C. to about 36° C., and most preferably from about 18° C. to about 33° C. If algae or yeast are used, a suitable temperature range for carrying out the cultivation may be for instance from about 15° C. to about 40° C., preferably from about 20° C. to about 45° C., more preferably from about 25° C. to about 40° C., even more preferably from about 25° C. to about 38° C., and most preferably from about 30° C. to about 38° C. The culture medium for growth usually may contain such nutrients as assimilable carbon sources, e.g., glycerol, D-mannitol, D-sorbitol, L-sorbose, erythritol, ribitol, xylitol, arabitol, inositol, dulcitol, D-ribose, D-fructose, sucrose, D-glucose or polymers thereof such as for example starch or maltose and the like; preferably L-sorbose, D-glucose, D-sorbitol, D-mannitol, and glycerol; and digestible nitrogen sources such as organic substances, e.g., peptone, yeast extract and amino acids. The media may be with or without urea and/or corn steep liquor and/or baker's yeast. Various inorganic substances may also be used as nitrogen sources, e.g., nitrates and ammonium salts. Furthermore, the growth medium usually may contain inorganic salts, e.g., magnesium sulfate, manganese sulfate, cupric sulfate, potassium phosphate, sodium phosphate, and calcium carbonate.

In connection with the above process, the specific growth rates are for instance at least 0.02 h⁻¹. For cells growing in batch, fed-batch or semi-continuous mode, the growth rate depends on for instance the composition of the growth medium, pH, temperature, and the like. In general, the growth rates may be for instance in a range from about 0.05 to about 0.2 h⁻¹, preferably from about 0.06 to about 0.15 h⁻¹, and most preferably from about 0.07 to about 0.13 h⁻¹.

In another aspect of the above process, resting cells may be provided by cultivation of the respective microorganism on agar plates thus serving as growth vessel, using essentially the same conditions, e.g., cultivation period, pH, temperature, nutrient medium as described above, with the addition of agar.

If the growth and production phase are performed in two separate vessels, then the cells from the growth phase may be harvested or concentrated and transferred to a second vessel, the so-called production vessel. This vessel may contain an aqueous medium supplemented with any applicable production substrate that can be converted to Hy-T by the cells. Cells from the growth vessel can be harvested or concentrated by any suitable operation, such as for instance centrifugation, membrane crossflow ultrafiltration or microfiltration, filtration, decantation, flocculation. The cells thus obtained may also be transferred to the production vessel in the form of the original broth from the growth vessel, without being harvested, concentrated or washed, i.e. in the form of a cell suspension. In a preferred embodiment, the cells are transferred from the growth vessel to the production vessel in the form of a cell suspension without any washing or isolation step in between.

If the growth and production phase are performed in the same vessel, cells may be grown under appropriate conditions to the desired cell density followed by a replacement of the growth medium with the production medium containing the production substrate. Such replacement may be, for instance, the feeding of production medium to the vessel at the same time and rate as the withdrawal or harvesting of supernatant from the vessel. To keep the resting cells in the vessel, operations for cell recycling or retention may be used, such as for instance cell recycling steps. Such recycling steps, for instance, include but are not limited to methods using centrifuges, filters, membrane crossflow microfiltration or ultrafiltration steps, membrane reactors, flocculation, or cell immobilization in appropriate porous, non-porous or polymeric matrixes. After a transition phase, the vessel is brought to process conditions under which the cells are in a resting cell mode as defined above, and the production substrate is efficiently converted into Hy-T.

Alternatively the cells could be used to produce Hy-T in growing mode such as when partially transforming a given substrate into Hy-T while partially using it as carbon source. Cells can be used as growing cells by supplying a carbon source and a substrate to be transformed into Hy-T or combinations of these. Cells can also be altered to be able to express the required activities upon induction by addition of external organic compounds (inducers).

The aqueous medium in the production vessel as used for the production step in connection with the above process using a microorganism, hereinafter called production medium, may contain only the production substrate(s) to be converted into Hy-T, or may contain for instance additional inorganic salts, e.g., sodium chloride, calcium chloride, magnesium sulfate, manganese sulfate, potassium phosphate, sodium phosphate, calcium phosphate, and calcium carbonate. The production medium may also contain digestible nitrogen sources such as for instance organic substances, e.g., peptone, yeast extract, urea, amino acids, and corn steep liquor, and inorganic substances, e.g. ammonia, ammonium sulfate, and sodium nitrate, at such concentrations that the cells are kept in a resting cell mode as defined above. The medium may be with or without urea and/or corn steep liquor and/or baker's yeast. The production step may be conducted for instance in batch, fed-batch, semi-continuous or continuous mode. In case of fed-batch, semi-continuous or continuous mode, both cells from the growth vessel and production medium can be fed continuously or intermittently to the production vessel at appropriate feed rates. Alternatively, only production medium may be fed continuously or intermittently to the production vessel, while the cells coming from the growth vessel are transferred at once to the production vessel. The cells coming from the growth vessel may be used as a cell suspension within the production vessel or may be used as for instance flocculated or immobilized cells in any solid phase such as porous or polymeric matrixes. The production period, defined as the period elapsed between the entrance of the substrate into the production vessel and the harvest of the supernatant containing Hy-T, the so-called harvest stream, can vary depending for instance on the kind and concentration of cells, pH, temperature and nutrient medium to be used, and is preferably about 2 to about 100 h. The pH and temperature can be different from the pH and temperature of the growth step, but is essentially the same as for the growth step.

In one embodiment, the production step is conducted in continuous mode, meaning that a first feed stream containing the cells from the growth vessel and a second feed stream containing the substrate is fed continuously or intermittently to the production vessel. The first stream may either contain only the cells isolated/separated from the growth medium or a cell suspension, coming directly from the growth step, i.e. cells suspended in growth medium, without any intermediate step of cell separation, washing and/or isolation and/or concentration. The second feed stream as herein defined may include all other feed streams necessary for the operation of the production step, e.g. the production medium comprising the substrate in the form of one or several different streams, water for dilution, and acid or base for pH control.

In connection with the above process, when both streams are fed continuously, the ratio of the feed rate of the first stream to feed rate of the second stream may vary between about 0.01 and about 10, preferably between about 0.01 and about 5, most preferably between about 0.02 and about 2. This ratio is dependent on the concentration of cells and substrate in the first and second stream, respectively.

Another way of performing the process as above using a microorganism of the present invention may be a process using a certain cell density of resting cells in the production vessel. The cell density is measured as absorbance units (optical density) at 600 nm by methods known to the skilled person. In a preferred embodiment, the cell density in the production step is at least about 2, more preferably between about 2 and about 200, even more preferably between about 10 and about 200, even more preferably between about 15 and about 200, even more preferably between about 15 to about 120, and most preferably between about 20 and about 120.

In order to keep the cells in the production vessel at the desired cell density during the production phase as performed, for instance, in continuous or semi-continuous mode, any means known in the art may be used, such as for instance cell recycling by centrifugation, filtration, membrane crossflow ultrafiltration or microfiltration, decantation, flocculation, cell retention in the vessel by membrane devices or cell immobilization. Further, in case the production step is performed in continuous or semi-continuous mode and cells are continuously or intermittently fed from the growth vessel, the cell density in the production vessel may be kept at a constant level by, for instance, harvesting an amount of cells from the production vessel corresponding to the amount of cells being fed from the growth vessel.

In connection with the above process, the produced Hy-T contained in the so-called harvest stream is recovered/harvested from the production vessel. The harvest stream may include, for instance, cell-free or cell-containing aqueous solution coming from the production vessel, which contains Hy-T as a result of the conversion of production substrate by the resting cells in the production vessel. Cells still present in the harvest stream may be separated from the Hy-T by any operations known in the art, such as for instance filtration, centrifugation, decantation, membrane crossflow ultrafiltration or microfiltration, tangential flow ultrafiltration or microfiltration or dead end filtration. After this cell separation operation, the harvest stream is essentially free of cells.

In a further aspect, the process of the present invention may be combined with further steps of separation and/or purification of the produced Hy-T from other components contained in the harvest stream, i.e., so-called downstream processing steps. These steps may include any means known to a skilled person, such as, for instance, concentration, extraction, crystallization, precipitation, adsorption, ion exchange, chromatography, distillation, electrodialysis, bipolar membrane electrodialysis and/or reverse osmosis. Any of these procedures alone or in combination constitute a convenient means for isolating and purifying the product, i.e. Hy-T. The product thus obtained may further be isolated in a manner such as, e.g. by concentration, crystallization, precipitation, washing and drying and/or further purified by, for instance, treatment with activated carbon, ion exchange and/or re-crystallization.

According to the invention, host cells that are altered to contain one or more genes capable of expressing an activity selected from the group defined above and exemplified herein are able to directly produce Hy-T from a suitable substrate in significantly higher yield, productivity, and/or efficiency than other known organisms.

Polynucleotides encoding enzymes as defined above and the selection thereof are hereinafter described in more detail. The term “gene” as used herein means a polynucleotide encoding a protein as defined above.

The invention encompasses polynucleotides as shown in SEQ ID NO: 1, SEQ ID NO: 3, SEQ ID NO:5, SEQ ID NO:7, SEQ ID NO:9, SEQ ID NO:11, SEQ ID NO:13, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32. SEQ ID NO:34, SEQ ID NO:36, SEQ ID NO:38 and SEQ ID NO:40.

The invention also encompasses polynucleotides which are substantially homologous to one of these sequences. In this context it should be mentioned that the expression of “a polynucleotide which is substantially homologous” refers to a polynucleotide sequence selected from the group consisting of:
a) polynucleotides encoding a protein comprising the amino acid sequence according to SEQ ID NO:2, SEQ ID NO:4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 and SEQ ID NO:41;
b) polynucleotides encoding a fragment or derivative of a polypeptide encoded by a polynucleotide of any of (a) wherein in said derivative one or more amino acid residues are conservatively substituted compared to said polypeptide, and said fragment or derivative has the activity of a HP or FG protein;
c) polynucleotides the complementary strand of which hybridizes under stringent conditions to a polynucleotide as defined in any one of (a) or (b) and which encode a HP or FG protein;
d) polynucleotides which are at least 70%, such as 85, 90 or 95% homologous to a polynucleotide as defined in any one of (a) to (c) and which encode a HP or FG polypeptide;
e) the complementary strand of a polynucleotide as defined in (a) to (d).

The invention also encompasses polypeptides as shown in SEQ ID NO: 2, SEQ ID NO: 4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 and SEQ ID NO:41.

The invention also encompasses polypeptides which are substantially homologous to one of these amino acid sequences. In this context it should be mentioned that the expression of “a polypeptide which is substantially homologous” refers to a polypeptide sequence selected from the group consisting of:
a) polypeptides comprising an amino acid sequence comprising a fragment or derivative of a polypeptide sequence according to SEQ ID NO: 2, SEQ ID NO: 4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, EQ ID NO:33; SEQ ID NO:35, SEQ ID NO:37, SEQ ID NO:39 and SEQ ID NO:41, and which have the activity of a HP or FG polypeptide;
b) polypeptides comprising an amino acid sequence encoded by a fragment or derivative of a polynucleotide sequence according to SEQ ID NO: 1, SEQ ID NO: 3, SEQ ID NO:5, SEQ ID NO:7, SEQ ID NO:9, SEQ ID NO:11, SEQ ID NO:13, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32; SEQ ID NO:34, SEQ ID NO:36, SEQ ID NO:38 and SEQ ID NO:40, and which have the activity of a HP or FG polypeptide;
c) polypeptides which are at least 50%, such as 70, 80 or 90% homologous to a polypeptide according to SEQ ID NO: 2, SEQ ID NO: 4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33; SEQ ID NO:35, SEQ ID NO:37, SEQ ID NO:39 and SEQ ID NO:41, or to a polypeptide according to (a) or (b) and which have the activity of a HP or FG polypeptide.

An “isolated nucleic acid fragment” is a nucleic acid fragment that is not naturally occurring as a fragment and would not be found in the natural state.

As used herein, the terms “polynucleotide”, “gene” and “recombinant gene” refer to nucleic acid molecules which may be isolated from chromosomal or plasmid DNA or may be generated by synthetic methods, which include an open reading frame (ORF) encoding a protein as exemplified above. A polynucleotide may include a polynucleotide sequence or fragments thereof and regions upstream and downstream of the gene sequences which may include, for example, promoter regions, regulator regions and terminator regions important for the appropriate expression and stabilization of the polypeptide derived thereof.

A gene may include coding sequences, non-coding sequences such as for instance untranslated sequences located at the 3′- and 5′-ends of the coding region of a gene, and regulatory sequences. Moreover, a gene refers to an isolated nucleic acid molecule as defined herein. It is furthermore appreciated by the skilled person that DNA sequence polymorphisms that lead to changes in the amino acid sequences of the protein may exist within a gene population. Such genetic polymorphism in the gene may exist among individuals within a population due to natural variation or in cells from different populations. Such natural variations can typically result in 1-5% variance in the nucleotide sequence of the corresponding gene. Any and all such nucleotide variations and the resulting amino acid polymorphism are the result of natural variation. They do not alter the functional activity of proteins and therefore they are intended to be within the scope of the invention.

As used herein, the terms “polynucleotide” or “nucleic acid molecule” are intended to include DNA molecules (e.g., cDNA or genomic DNA) and RNA molecules (e.g., mRNA) and analogs of the DNA or RNA generated using nucleotide analogs. The nucleic acid molecule may be single-stranded or double-stranded, but preferably is double-stranded DNA. The nucleic acid may be synthesized using oligonucleotide analogs or derivatives (e.g., inosine or phosphorothioate nucleotides). Such oligonucleotides may be used, for example, to prepare nucleic acids that have altered base-pairing abilities or increased resistance to nucleases.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence may be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

Homologous or substantially identical gene sequences may be isolated, for example, by performing PCR using two degenerate oligonucleotide primer pools designed on the basis of nucleotide sequences as taught herein.

The template for the reaction may be cDNA obtained by reverse transcription of mRNA prepared from strains known or suspected to express a polynucleotide according to the invention. The PCR product may be subcloned and sequenced to ensure that the amplified sequences represent the sequences of a new nucleic acid sequence as described herein, or a functional equivalent thereof.

The PCR fragment may then be used to isolate a full length cDNA clone by a variety of known methods. For example, the amplified fragment may be labelled and used to screen a bacteriophage or cosmid cDNA library. Alternatively, the labelled fragment may be used to screen a genomic library.

PCR technology can also be used to isolate full-length cDNA sequences from other organisms. For example, RNA may be isolated, following standard procedures, from an appropriate cellular or tissue source. A reverse transcription reaction may be performed on the RNA using an oligonucleotide primer specific for the most 5′-end of the amplified fragment for the priming of first strand synthesis.

The resulting RNA/DNA hybrid may then be “tailed” (e.g., with guanines) using a standard terminal transferase reaction, the hybrid may be digested with RNaseH, and second strand synthesis may then be primed (e.g., with a poly-C primer). Thus, cDNA sequences upstream of the amplified fragment may easily be isolated. For a review of useful cloning strategies, see e.g., Sambrook, et al. (Sambrook J. et al. “Molecular Cloning: A Laboratory Manual” Cold Spring Harbor (NY, USA): Cold Spring Harbor Laboratory Press, 2001); and Ausubel et al. (Ausubel F. M. et al., “Current Protocols in Molecular Biology”, John Wiley & Sons (NY, USA): John Wiley & Sons, 2007).

Homologues, substantially identical sequences, functional equivalents, and orthologs of genes and proteins exemplified herein, such as for example the gene according to SEQ ID NO:5, and the encoded protein according to SEQ ID NO:6, may be obtained from a number of different microorganisms. In this context it should be mentioned that also the following paragraphs apply mutatis mutandis for all other enzymes defined above.

The procedures for the isolation of specific genes and/or fragments thereof are exemplified herein. Accordingly, nucleic acids encoding other family members, which thus have a nucleotide sequence that differs from a nucleotide sequence according to SEQ ID NO:5, are within the scope of the invention. Moreover, nucleic acids encoding proteins from different species which thus have a nucleotide sequence which differs from a nucleotide sequence shown in SEQ ID NO:5 are within the scope of the invention.

The invention also discloses an isolated polynucleotide hybridisable under stringent conditions, preferably under highly stringent conditions, to a polynucleotide according to the present invention, such as for instance a polynucleotide shown in SEQ ID NO:5. Advantageously, such polynucleotide may be obtained from a microorganism capable of converting a given carbon source directly into Hy-T.

As used herein, the term “hybridizing” is intended to describe conditions for hybridization and washing under which nucleotide sequences at least about 50%, at least about 60%, at least about 70%, more preferably at least about 80%, even more preferably at least about 85% to 90%, most preferably at least 95% homologous to each other typically remain hybridized to each other.

A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° C., more preferably at 60° C. and even more preferably at 65° C.

Highly stringent conditions include, for example, 2 h to 4 days incubation at 42° C. using a digoxigenin (DIG)-labelled DNA probe (prepared by using a DIG labeling system; Roche Diagnostics GmbH, 68298 Mannheim, Germany) in a solution such as DigEasyHyb solution (Roche Diagnostics GmbH) with or without 100 μg/ml salmon sperm DNA, or a solution comprising 50% formamide, 5×SSC (150 mM NaCl, 15 mM trisodium citrate), 0.02% sodium dodecyl sulfate, 0.1% N-lauroylsarcosine, and 2% blocking reagent (Roche Diagnostics GmbH), followed by washing the filters twice for 5 to 15 minutes in 2×SSC and 0.1% SDS at room temperature and then washing twice for 15-30 minutes in 0.5×SSC and 0.1% SDS or 0.1×SSC and 0.1% SDS at 65-68° C.

The skilled artisan will know which conditions to apply for stringent and highly stringent hybridization conditions. Additional guidance regarding such conditions is readily available in the art, for example, in Sambrook et al., (supra), Ausubel et al. (supra). Of course, a polynucleotide which hybridizes only to a poly (A) sequence (such as the 3′-terminal poly (A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A nucleic acid molecule of the present invention, such as for instance a nucleic acid molecule shown in SEQ ID NO:5 or a fragment or derivative thereof, may be isolated using standard molecular biology techniques and the sequence information provided herein. For example, using all or portion of the nucleic acid sequence shown in SEQ ID NO:5 as a hybridization probe, nucleic acid molecules according to the invention may be isolated using standard hybridization and cloning techniques (e.g., as described in Sambrook et al. (supra)).

Furthermore, oligonucleotides corresponding to or hybridisable to nucleotide sequences according to the invention may be prepared by standard synthetic techniques, e.g., using an automated DNA synthesizer, or delivered by gene synthesis as carried out by companies such as, for example, DNA2.0 (DNA2.0, Menlo Park, 94025 CA, USA) based on the sequence information provided herein.

The terms “homology”, “identically”, “percent identity” or “similar” are used interchangeably herein. For the purpose of this invention, it is defined here that in order to determine the percent identity of two amino acid sequences or of two nucleic acid sequences, the sequences are aligned for optimal comparison purposes (e.g., gaps may be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

The skilled person will be aware of the fact that several different computer programs are available to determine the homology between two sequences. For instance, a comparison of sequences and determination of percent identity between two sequences may be accomplished using a mathematical algorithm. In a preferred embodiment, the percent identity between two amino acid sequences is determined using the Needleman and Wunsch algorithm (Needleman and Wunsch, J. Mol. Biol. (1970) 48:443-453) which has been incorporated into the GAP program in the GCG software package (available at http://www.accelrys.com), using either a BLOSUM62 matrix or a PAM250 matrix, and a gap weight of 16, 14, 12, 10, 8, 6 or 4 and a length weight of 1, 2, 3, 4, 5 or 6. The skilled person will appreciate that all these different parameters will yield slightly different results but that the overall percentage identity of two sequences is not significantly altered when using different algorithms.

In yet another embodiment, the percent identity between two or more nucleotide sequences is determined using the GAP or ClustalW+ programs in the GCG software package (available at http://www.accelrys.com), using for example a NWSGAPDNA.CMP matrix and a gap weight of 40, 50, 60, 70 or 80 and a length weight of 1, 2, 3, 4, 5 or 6. In another embodiment, the percent identity between two amino acid or nucleotide sequences is determined using the algorithm of E. Meyers and W. Miller (Meyers and Miller, Comput. Appl. Biosci. (1989) 4:11-17) which has been incorporated into the ALIGN program (version 2.0) (available at http://vega.igh.cnrs.fr/bin/align-guess.cgi) using a PAM120 weight residue table, a gap length penalty of 12 and a gap penalty of 4.

The nucleic acid and protein sequences of the present invention may further be used as a “query sequence” to perform a search against public databases to, for example, identify other family members or related sequences. Such searches may be performed using the BLASTN and BLASTP programs (version 2.0) of Altschul, et al. (J. Mol. Biol. (1990) 215:403-410). BLAST nucleotide searches may be performed with the BLASTN program, score=100, word length=12 to obtain nucleotide sequences homologous to the nucleic acid molecules of the present invention. BLAST protein searches may be performed with the BLASTP program, score=50, word length=3 to obtain amino acid sequences homologous to the protein molecules of the present invention. To obtain gapped alignments for comparison purposes, Gapped BLAST may be utilized as described in Altschul et al., (Nucleic Acids Res. (1997) 25:3389-3402). When utilizing BLAST and Gapped BLAST programs, the default parameters of the respective programs (e.g., BLASTP and BLASTN) may be used (see for example http://www.ncbi.nim.nih.gov).

In another embodiment, an isolated nucleic acid molecule of the invention comprises a nucleic acid molecule which is the complement of a nucleotide sequence as of the present invention, such as for instance the sequence shown in SEQ ID NO:5. A nucleic acid molecule, which is complementary to a nucleotide sequence disclosed herein, is one that is sufficiently complementary to a nucleotide sequence shown in SEQ ID NO:5 such that it may hybridize to said nucleotide sequence thereby forming a stable duplex.

In a further embodiment, a nucleic acid of the invention, as for example shown in SEQ ID NO:5, or the complement thereof contains at least one mutation leading to a gene product with modified function/activity. The at least one mutation may be introduced by methods known in the art or described herein. In regard to the group of enzymes exemplified herein above, the at least one mutation leads to a protein whose function compared to the wild type counterpart is enhanced or improved. The activity of the protein is thereby increased. Methods for introducing such mutations are well known in the art.

Another aspect pertains to vectors, containing a nucleic acid encoding a protein according to the invention or a functional equivalent or portion thereof. As used herein, the term “vector” refers to a nucleic acid molecule capable of transporting another nucleic acid to which it has been linked. One type of vector is a “plasmid”, which refers to a circular double stranded DNA molecule into which additional DNA segments may be incorporated. Another type of vector is a viral vector, wherein additional DNA segments may be inserted into the viral genome. Certain vectors are capable of autonomous replication in a host cell into which they are introduced (e.g., bacterial vectors having an origin of DNA replication that is functional in said bacteria). Other vectors are integrated into the genome of a host cell upon introduction into the host cell, and thereby are replicated along with the host genome.

Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention may be designed for expression of enzymes as defined above in a suitable microorganism. Expression vectors useful in the present invention include chromosomal-, episomal- and virus-derived vectors e.g., vectors derived from bacterial plasmids, bacteriophage, and vectors derived from combinations thereof, such as those derived from plasmid and bacteriophage genetic elements, such as cosmids and phagemids.

The recombinant vectors of the invention comprise a nucleic acid of the invention in a form suitable for expression of the nucleic acid in a host cell, which means that the recombinant expression vector includes one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., attenuators). Such regulatory sequences are described, for example, in “Methods in Enzymology”, Volume 185: “Gene Expression Technology”, Goeddel D V (Ed.), Academic Press (San Diego, Calif.), 1990. Regulatory sequences include those which direct constitutive or inducible expression of a nucleotide sequence in many types of host cells and those which direct expression of the nucleotide sequence only in a certain host cell (e.g. tissue-specific regulatory sequences). It will be appreciated by those skilled in the art that the design of the expression vector can depend on such factors as the choice of the host cell to be transformed, the level of expression of protein desired, etc. The expression vectors of the invention may be introduced into host cells to thereby produce proteins or peptides, encoded by nucleic acids as described herein, including, but not limited to, mutant proteins, fragments thereof, variants or functional equivalents thereof, and fusion proteins, encoded by a nucleic acid as described herein.

The DNA insert may be operatively linked to an appropriate promoter, which may be either a constitutive or inducible promoter. The skilled person will know how to select suitable promoters. The expression constructs may contain sites for transcription initiation, termination, and, in the transcribed region, a ribosome binding site for translation. The coding portion of the mature transcripts expressed by the constructs may preferably include an initiation codon at the beginning and a termination codon appropriately positioned at the end of the polypeptide to be translated.

Vector DNA may be introduced into suitable host cells via conventional transformation or transfection techniques. As used herein, the terms “transformation”, “conjugation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, transduction, infection, lipofection, cationic lipid-mediated transfection or electroporation. Suitable methods for transforming or transfecting host cells may be found in Sambrook, et al. (supra), Davis et al., (“Basic Methods in Molecular Biology”, Elsevier (NY, USA), 1986) and other laboratory manuals.

In order to identify and select cells which have integrated the foreign DNA into their genome, a gene that encodes a selectable marker (e.g., resistance to antibiotics) is generally introduced into the host cells along with the gene of interest. Preferred selectable markers include those that confer resistance to drugs, such as kanamycin, tetracycline, ampicillin and streptomycin. A nucleic acid encoding a selectable marker is preferably introduced into a host cell on the same vector as that encoding a protein according to the invention or can be introduced on a separate vector such as, for example, a suicide vector, which cannot replicate in the host cells. Cells stably transfected with the introduced nucleic acid can be identified by drug selection (e.g., cells that have incorporated the selectable marker gene will survive, while the other cells die).

As mentioned above, the polynucleotides of the present invention may be utilized in the genetic engineering of a suitable host cell to make it better and more efficient in the production, for example in a direct fermentation process, of Hy-T.

Therefore, the invention also relates to the concurrent use of genes encoding polypeptides having activities as specified above. Such a host cell will then show an improved capability to directly produce Hy-T.

The alteration in the genome of the microorganism may be obtained e.g. by replacing through a single or double crossover recombination a wild type DNA sequence by a DNA sequence containing the alteration. For convenient selection of transformants of the microorganism with the alteration in its genome the alteration may, e.g. be a DNA sequence encoding an antibiotic resistance marker or a gene complementing a possible auxotrophy of the microorganism. Mutations include, but are not limited to, deletion-insertion mutations.

An alteration in the genome of the microorganism leading to a more functional polypeptide may also be obtained by randomly mutagenizing the genome of the microorganism using e.g. chemical mutagens, radiation or transposons and selecting or screening for mutants which are better or more efficient producers of one or more fermentation products. Standard methods for screening and selection are known to the skilled person.

In another specific embodiment, it is desired to enhance and/or improve the activity of a protein selected from the group of enzymes specified herein above.

The invention also relates to microorganisms wherein the activity of a given polypeptide is enhanced and/or improved so that the yield of Hy-T which is directly produced is increased, preferably in those organisms that overexpress the said polypeptides or an active fragment or derivative thereof. This may be accomplished, for example, by transferring a polynucleotide according to the invention into a recombinant or non-recombinant microorganism that may or may not contain an endogenous equivalent of the corresponding gene.

The skilled person will know how to enhance and/or improve the activity of a protein. Such may be accomplished by either genetically modifying the host organism in such a way that it produces more or more stable copies of the said protein than the wild type organism. It may also be accomplished by increasing the specific activity of the protein.

In the following paragraphs procedures are described how to achieve this goal, i.e. the increase in the yield and/or production of Hy-T by increasing (up-regulation) the activity of a specific protein. These procedures apply mutatis mutandis for the similar proteins whose functions, compared to the wild type counterpart, have to be enhanced or improved.

Modifications in order to have the organism produce more copies of specific gene, i.e. overexpressing the gene, and/or protein may include the use of a strong promoter, or the mutation (e.g. insertion, deletion or point mutation) of (parts of) the gene or its regulatory elements. It may also involve the insertion of multiple copies of the gene into a suitable microorganism. An increase in the specific activity of a protein may also be accomplished by methods known in the art. Such methods may include the mutation (e.g. insertion, deletion or point mutation) of (parts of) the encoding gene.

A mutation as used herein may be any mutation leading to a more functional or more stable polypeptide, e.g. more functional or more stable gene products. This may include for instance an alteration in the genome of a microorganism, which improves the synthesis of the protein or leads to the expression of the protein with an altered amino acid sequence whose function compared with the wild type counterpart having a non-altered amino acid sequence is improved and/or enhanced. The interference may occur at the transcriptional, translational or post-translational level.

The term “increase” of activity as used herein encompasses increasing activity of one or more polypeptides in the producing organism, which in turn are encoded by the corresponding polynucleotides described herein. There are a number of methods available in the art to accomplish the increase of activity of a given protein. In general, the specific activity of a protein may be increased or the copy number of the protein may be increased.

To facilitate such an increase, the copy number of the genes corresponding to the polynucleotides described herein may be increased. Alternatively, a strong promoter may be used to direct the expression of the polynucleotide. In another embodiment, the promoter, regulatory region and/or the ribosome binding site upstream of the gene can be altered to increase the expression. The expression may also be enhanced or increased by increasing the relative half-life of the messenger RNA. In another embodiment, the activity of the polypeptide itself may be increased by employing one or more mutations in the polypeptide amino acid sequence, which increases the activity. For example, lowering the relative Km and/or increasing the kcat of the polypeptide with its corresponding substrate will result in improved activity. Likewise, the relative half-life of the polypeptide may be increased. In either scenario, that being enhanced gene expression or increased specific activity, the improvement may be achieved by altering the composition of the cell culture medium and/or methods used for culturing. “Enhanced expression” or “improved activity” as used herein means an increase of at least 5%, 10%, 25%, 50%, 75%, 100%, 200% or even more than 500%, compared to a wild-type protein, polynucleotide, gene; or the activity and/or the concentration of the protein present before the polynucleotides or polypeptides are enhanced and/or improved. The activity of the protein may also be enhanced by contacting the protein with a specific or general enhancer of its activity.

The invention is further illustrated by the following examples which should not be construed as limiting.

Materials and Methods

Strains and Plasmids

Bacterial strains used for the invention were Escherichia coli W (ATCC 11105, American Type Culture Collection), Escherichia coli DH10B, Escherichia coli TOP10 (Invitrogen), Escherichia coli MG1655 (CGSC No. 7740, E. coli Genetic Stock Center), Acinetobacter calcoaceticus EBF 65/61 (Barrowman M. M. and Fewson C. A. Curr. Microbiol. (1985) 12:235-240), Pseudomonas putida U, Pseudomonas putida A7 (Olivera E. R. et al. Eur. J. Biochem. (1994) 221:375-381), Pseudomonas putida KT2440 (DSMZ 6125, German Collection of Microorganisms and Cell Cultures), Rhodococcus erythropolis (DSMZ 43297, German Collection of Microorganisms and Cell Cultures). Plasmids used in this study were pCR-XL-TOPO (Invitrogen), pZErO-2 (Invitrogen), pCK01, pUC18, pJF119EH (Furste et al., Gene (1986) 48: 119-131) and pJF119EH hpaB hpaC (also referred to as pJF hpaB hpaC, pJFhpaBC, or pD1). Plasmid pJF119EH hpaB hpaC (alias pD1) is described in WO 2004/015094 and was deposited under the Budapest Treaty on 23 Jul. 2002 with the DSMZ under number DSM 15109.

TABLE 1
Description of strains and plasmids used for hydroxytyrosol production

| Host Strain & | |
|---|---|
| Plasmids | Description |
| E. coli TOP10 | F⁻ mcrA Δ(mrr⁻hsdRMS⁻mcrBC) φ80lacZΔM15 ΔlacX74 deoR recA1 endA1 |
| | araΔ139 Δ(ara, leu)7697 galU galK λ- rpsL(StrR) nupG. |
| pD1 = pJFhpaBC | hpaBC genes coding for 4-hydroxyphenylacetic acid 3-monooxygenase from |
| | E. coli W ATCC 11105 cloned as a BamHI/HindIII fragment in the MCS of |
| | vector pJF119EH under the control of an IPTG-inducible tac promoter; Ap^{R}. |
| pPH | palR ORF coding for phenylacetaldehyde reductase from Rhodococcus |
| | erythropolis (DSMZ 43297) cloned as a SmaI/BamHI fragment in plasmid |
| | pD1 under the control of an IPTG-inducible tac promoter; Ap^{R}. |
| pMPH | maoA ORF coding for monoamine oxidase from E. coli MG1655 (CGSC # |
| | 7740) cloned as a EcoRI/SmaI fragment in in plasmid pPH under the control |
| | of an IPTG-inducible tac promoter; Ap^{R}. |
| pDMPH | tyrD codon optimized synthetic gene (DNA 2.0) coding for L-tyrosine |
| | decarboxylase from Methanocaldococcus jannaschii cloned as a EcoRI/KpnI |
| | fragment in plasmid pMPH under the control of an IPTG-inducible tac |
| | promoter; Ap^{R}. |

General Microbiology

All solutions were prepared in deionized water. LB medium (1 L) contained Bacto tryptone (10 g), Bacto yeast extract (5 g), and NaCl (10 g). 2*TY medium (1 L) contained Bacto tryptone (16 g), Bacto yeast extract (10 g) and NaCl (5 g). Nutrient broth (1 L) contained peptone (5 g) and meat extract (3 g). M9 salts (1 L) contained Na₂HPO₄ (6 g), KH₂PO₄ (3 g), NH₄Cl (1 g), and NaCl (0.5 g). M9 medium contained D-glucose (4 g) and MgSO₄ (1 mM) in 1 L of M9 salts. M9 inoculation medium contained D-glucose (4 g), casamino acids (20 g) and MgSO₄ (1 mM) in 1 L of M9 salts. M9 induction medium contained D-glucose (40 g), casamino acids (20 g) and MgSO₄ (1 mM) in 1 L of M9 salts. Unless stated otherwise, antibiotics were added where appropriate to the following final concentrations: ampicillin (Ap), 100 mg/L; kanamycin (Km), 50 mg/L; chloramphenicol (Cm), 33 mg/L. Casamino acids (Difco cat. no. 223120) were prepared as 20% stock solution in water. Stock solutions of 4-hydroxyphenylacetic acid (405 mM), tyrosol (405 mM), tyramine (810 mM) were prepared in potassium phosphate buffer (50 mM, pH 7.0); L-tyrosine (0.2-0.3 M) was titrated into solution using KOH. Isopropyl-β-D-thiogalactopyrano side (IPTG) was prepared as a 100 mM stock solution in water. Solutions of LB medium, M9 salts, MgSO₄, and D-glucose were autoclaved individually prior to mixing. Copper(II) sulphate (CuSO₄) was prepared as a 50 mM stock solution in water and added to bacterial cells as specified in the text. Solutions of antibiotics, casamino acids, tyrosol, 4-hydroxyphenylacetic acid, tyramine, L-tyrosine, ascorbic acid, glycerol, IPTG and CuSO₄ were sterilized through 0.22-μm membranes. Solid medium was prepared by addition of Difco agar to a final concentration of 1.5% (w/v). Unless otherwise stated, liquid cultures of E. coli were grown at 37° C. with agitation at 250 rpm and solid cultures were incubated at 30° C. Bacterial growth was monitored by measuring the optical density (O.D.) of liquid cultures at 600 nm (OD₆₀₀) using a spectrophotometer. Standard molecular cloning techniques well known to those skilled in the art were performed for construction and analysis of plasmid DNA as well as for transformation of E. coli strains as described in Sambrook J. et al. “Molecular Cloning: A Laboratory Manual” Cold Spring Harbor (NY, USA): Cold Spring Harbor Laboratory Press, 2001. Commercially available kits for the isolation and amplification of nucleic acids were used according to manufacturer's instructions. QIAprep Spin Miniprep Kit was purchased from Qiagen and used for plasmid DNA isolation. High Pure PCR Template Preparation Kit was purchased from Roche Diagnostics and used for chromosomal DNA isolation. Polymerase chain reactions (PCR) were performed with Herculase™ Enhanced DNA Polymerase from Stratagene using iCycler, a thermal cycler from BioRad. Restriction enzymes were purchased from New England Biolabs or Roche Diagnostics. Nucleic acid ligations were performed using T4 ligase from Roche Diagnostics.

Preparation of Working Cell Banks

Inoculants of E. coli strains were started by introducing one single colony picked off a freshly streaked agar plate into 5 mL of M9 inoculation medium containing the appropriate antibiotic. Cultures were grown for 24 h then used to inoculate 50 mL of M9 induction medium containing the appropriate antibiotic to a starting OD₆₀₀ of 0.025-0.05 (1% inoculum). The 50 mL culture was grown at 37° C. with agitation at 250 rpm to OD₆₀₀=0.4-0.6 then used to prepare several frozen cell stocks in 20% glycerol (up to 27 cryovials per culture). Typically, 0.75 mL cell suspension was aseptically mixed with 0.25 mL 80% glycerol then stocked at −80° C. until used.

NMR Analysis

Phenylpyruvate decarboxylase activity was screened and assayed by proton nuclear magnetic resonance (¹H-NMR) spectroscopy detection of phenylacetate production with concomitant phenylpyruvate consumption as described by Sonke T. et al. “Industrial Perspectives on Assays”, in “Enzyme Assays: High-throughput Screening, Genetic Selection and Fingerprinting”, edited by Reymond J.-L. Weinheim (Germany): Wiley-VCH, 2006, p. 95-136.

TLC Analysis

Thin layer chromatograpy (TLC) analysis of L-tyrosine decarboxylase activity was performed as described by Garcia-Moruno E. et al. J. Food Prot. (2005) 68:625-629 using a mixture of chloroform:triethanolamine (100:1, v/v) as mobile phase to separate dansyl derivatives.

HPLC Analysis

Reactions were sampled (1.0 mL) at several time-points during the cultivation or incubation period. Samples were centrifuged to remove cells debris. The clear supernatant (0.75 mL) was transferred to an amber glass vial for HPLC analysis. Reverse phase HPLC methods were developed for the simultaneous quantification of tyrosol, hydroxytyrosol, 4-hydroxyphenylacetic acid, 3,4-dihydroxyphenylacetic acid, tyramine, L-tyrosine and related substances (see below): Method 2 results in a better resolution of L-tyrosine and tyramine compared to Method 1 (Table 2). HPLC was performed on an Agilent 1100 HPLC system equipped with a thermostatic autosampler and a diode array detector. The separation was carried out using a Phenomenex Security Guard C18 guard column (4 mm×3.0 mm I.D.) and a YMC Pack ProC18 analytical column (5 μm, 150 mm×4.6 mm I.D.). The column temperature was maintained at 23° C. and the flow rate at 1.0 mL/min. Typically, the column pressure varied from 70 (at start) to 120 bar. Sample detection was achieved at 210 nm. The injection volume was 3 μL. Compounds were identified by comparison of retention times and their online-recorded UV spectra with those of reference compounds. Concentrations were calculated by integration of peak areas and based on previously constructed standard calibration curves (see Table 2 for list of retention times).

Method 1: a gradient of acetonitrile (ACN) in 0.1% aqueous methanesulfonic acid was used as a mobile phase with the following elution profile: 0 to 5 min, 10% ACN; 5 to 20 min, increase ACN to 90%; 20 to 25 min, hold ACN at 90%.

Method 2: a gradient of ACN in 0.1% aqueous methanesulfonic acid was used as a mobile phase with the following elution profile: 0 to 3 min, 6% ACN; 4 to 20 min, increase ACN to 70%; 20 to 25 min, hold ACN at 70%.

TABLE 2
HPLC retention times

| | | Retention Time (min) | |
|---|---|---|---|
| | Compound | Method 1 | Method 2 |
| Compound Name | Abbreviation | (old) | (new) |
| Dopamine | Dopa-NH2 | 1.75 | 2.12 |
| Tyramine | Tyr-NH2 | 2.03 | 2.50 |
| L-Tyrosine | Tyr | 2.19 | 2.92 |
| L-Phenylalanine | Phe | 3.25 | 5.10 |
| 2-Phenylethylamine | Phe-NH2 | 3.60 | 5.71 |
| Hydroxytyrosol | HO-Tyrosol | 4.80 | 7.65 |
| 3,4-Dihydroxyphenylacetic acid | 3,4-DHPA | 6.50 | 9.11 |
| Tyrosol | 4-HPE | 7.80 | 10.00 |
| 4-Hydroxyphenylacetic acid | 4-HPA | 9.59 | 11.35 |
| 2-(3-Hydroxyphenyl)ethanol | 3-HPE | 9.63 | 11.39 |
| 2-Phenylethanol | 2-PE | 12.7 | 13.29 |
| 4-Methoxyphenylacetic acid | 4-MEPA | 13.3 | 15.57 |

### EXAMPLES OF HYDROXYTYROSOL PRODUCTION FROM TYROSOL

### Example 1

### Bioconversion of Tyrosol to Hydroxytyrosol by Non-Pathogenic Escherichia coli Strains

The non-pathogenic microorganism Escherichia coli W ATCC 11105 was tested for its ability to transform tyrosol into hydroxytyrosol (Prieto M. A. and García J. L. Biochem. Biophys. Res. Comm. (1997) 232:759-765). Expression of chromosomal hpa genes such as hpaB and hpaC, encoding the two-component flavin diffusible 4-hydroxyphenylacetate 3-monooxygenase, could be induced by adding phenylacetic acid and/or molecules derived therefrom, such as for example 4-hydroxyphenylacetic acid or 3-hydroxyphenylacetic acid, to the cell culture medium. A single colony of E. coli W picked off a plate of solidified LB medium was used to inoculate 50 mL of LB broth. The resulting culture was incubated overnight at 37° C. with shaking at 250 rpm to ensure proper aeration. The overnight growth was used to inoculate each of two 50 mL cultures of fresh LB broth to an optical density (O.D.) at 600 nm of 0.1. Cultivation was resumed under the same conditions until an O.D. at 600 nm of 0.5 was reached. At this point, hpaBC gene expression was induced by adding 1 mM 4-hydroxyphenylacetic acid to one of the cultures. The second culture was left untreated to provide E. coli W control cells that do not express hpaBC genes. Growth was resumed for another 3.5 hours. Cells were harvested by centrifugation, washed with 5 mL of potassium phosphate buffer (50 mM, pH 7.0), and finally resuspended in fresh buffer to a final O.D. of 20-40. Varying amounts of cell suspension (0.25-3.0 mL) were set up in biotransformation reactions (5 mL) in the presence of tyrosol (16 mM) and ascorbic acid (40 mM) in potassium phosphate buffer (50 mM, pH 7.0). The reactions were incubated at 37° C. with shaking at 250 rpm to ensure proper aeration. Samples were withdrawn and the advancement of the reaction monitored by HPLC analysis of the cell-free supernatants as described in the Materials and Methods section. After 18 h reaction time, hydroxytyrosol was obtained with up to 26% yield (mol/mol from tyrosol) in reactions containing induced E. coli W cells to an O.D. at 600 nm of 20. E. coli W cells that remained untreated with inducer 4-hydroxyphenylacetic acid during cultivation did not catalyze the formation of hydroxytyrosol from tyrosol. Our observations demonstrate that upregulated hpaBC gene expression results in tyrosol conversion into hydroxytyrosol by E. coli W ATCC 11105 cells. To date, the ability of microorganisms to convert tyrosol into hydroxytyrosol was always associated with their ability to utilize tyrosol as the sole carbon and energy source for growth (Allouche N. et al. Appl. Environ. Microbiol. (2004) 70:2105-2109 and J. Agric. Food. Chem. (2005) 53:6525-6530), but the enzymes or encoding genes that catalyze the formation of hydroxytyrosol itself had not been identified so far. No E. coli strain was ever described as able to grow on tyrosol as sole carbon and energy source (Diaz E. et al. Microbiol. Mol. Biol. Rev. (2001) 65:523-569). The discovery that an E. coli strain such as E. coli W ATCC 11105 is capable of tyrosol-to-hydroxytyrosol conversion was therefore unexpected. Also unexpected was the clear identification of the enzyme 4-hydroxyphenylacetate 3-monooxygenase (HpaBC) and encoding genes hpaB and hpaC as responsible for hydroxytyrosol formation from tyrosol.

### Example 2

### Bioconversion of Tyrosol to Hydroxytyrosol by Resting Escherichia coli Cells Expressing hpaB and hpaC Genes

The hpaB (SEQ ID NO:5) and hpaC (SEQ ID NO: 7) open reading frames (ORFs) from E. coli W ATCC 11105, encoding a 4-hydroxyphenylacetate 3-hydroxylase (SEQ ID NO:6) and a flavin:NAD(P)H reductase (SEQ ID NO:8), respectively, were made available as described by Kramer M. et al. WO 2004/015094. In the resulting plasmid pD1, hpaBC genes are transcribed from the IPTG-inducible tac promoter. Competent cells of E. coli strain TOP10 (Invitrogen), an E. coli K-12 derivative lacking hpa genes, were transformed with plasmid pD1. The resulting recombinant E. coli strain TOP10/pD1 was tested for its ability to convert tyrosol to hydroxytyrosol. Inoculants were started from one single colony of E. coli TOP10/pD1 and grown overnight at 37° C. with agitation at 250 rpm in LB broth (5 mL) containing ampicillin (100 mg/L). An aliquot of overnight culture (1% inoculum) was transferred to fresh LB broth (25 mL) containing ampicillin (100 mg/mL). The culture was grown at 37° C. with agitation at 250 rpm to OD₆₀₀=0.5, at which point protein expression was induced by adding IPTG to a final concentration of 1 mM. Cultivation was resumed until an OD₆₀₀ of 1.0 was reached. Cells were harvested by centrifugation (3220 g, 15 min) then resuspended in 5 mL of Tris-HCl buffer (10 mM, pH 8.0). Aliquots (1 mL) were dispensed in three separate reaction tubes: tube no. 1 was treated with tyrosol (5 mM); tube no. 2 was treated with 4-hydroxyphenylacetic acid (5 mM) to provide a positive control; tube no. 3 was left untreated to provide a negative control. After 48 h incubation at 37° C. with shaking at 350 rpm, only tubes no. 1 and 2 presented a brown coloration indicative of the formation of catechol derivatives. The formation of hydroxytyrosol from tyrosol in tube no. 1 was confirmed by TLC analysis. Resting cells of E. coli TOP10/pD1 expressing plasmid-encoded hpaBC genes catalyzed the formation of hydroxytyrosol from tyrosol in a 20% conversion ratio as judged by ¹H-NMR analysis of the cell-free reaction supernatant. This experiment demonstrates tyrosol hydroxylase activity for the hpaB- and hpaC-encoded enzyme HpaBC. A person skilled in the art will recognize that numerous microorganisms other than E. coli which are able to metabolize 4-hydroxyphenylacetic acid or related aromatic molecules, would also be expected to produce hydroxytyrosol via aromatic hydroxylation regardless of whether or not these microorganisms are able to utilize tyrosol or hydroxytyrosol as a carbon and energy source.

### Example 3

### Bioconversion of 2-(3-hydroxyphenyl)ethanol to Hydroxytyrosol by Resting Escherichia coli Cells Expressing hpaB and hpaC Genes

Inoculants were started from one single colony of E. coli TOP10/pD1 and grown overnight at 37° C. with agitation at 250 rpm in LB broth (5 mL) containing ampicillin (100 mg/L). An aliquot of overnight culture was transferred to each of two cultures of fresh LB broth (50 mL) containing ampicillin (100 mg/mL). Both cultures were grown at 37° C. with agitation at 250 rpm to OD₆₀₀=0.85, at which point protein expression was induced in one of the cultures by adding IPTG to a final concentration of 0.5 mM. The other culture was left untreated to provide cells for negative controls. Cultivation was resumed for 3 h at 37° C. with shaking. Cells were harvested by centrifugation (2500 g, 10 min), washed in 5 mL potassium phosphate buffer (50 mM, pH 7.0), then resuspended in 8 mL of that same buffer to final OD₆₀₀=11 for control cells, and OD₆₀₀=10.5 for IPTG-treated cells. Aliquots (1 mL) were dispensed in separate reaction tubes: tubes 1a, 2a, and 3a contained control cells; tubes 1b, 2b, and 3b contained IPTG-treated E. coli TOP10/pD1 cells; tubes 1a and 1b were treated with ethanol (0.1 mL) to provide a negative control; tubes 2a and 2b were treated with tyrosol (15 mM) to provide a positive control; and tubes 3a and 3b were treated with 2-(3-hydroxyphenyl)ethanol (25 mM). Reactions were incubated for 20 h at 37° C. with shaking at 250 rpm. Only tubes 2b and 3b presented a brown coloration indicative of the formation of catechol derivatives such as hydroxytyrosol. No hydroxytyrosol as detected by HPLC analysis in negative control reactions 1a or 1b treated with ethanol. As a positive control, HPLC analysis of reactions 2a and 2b cell-free supernatants confirmed that the production of hydroxytyrosol from tyrosol was higher in reactions containing IPTG-induced E. coli TOP10/pD1 cells (up to 26% molar conversion ratio) as compared to reactions containing control E. coli TOP10/pD1 cells (less than 4% molar conversion ratio). HPLC analysis of reactions 3a and 3b demonstrated that resting cells of E. coli TOP10/pD1 expressing plasmid-encoded hpaBC genes catalyzed the production of hydroxytyrosol from a source other than tyrosol: reactions containing IPTG-induced E. coli TOP10/pD1 cells showed a 2-(3-hydroxyphenyl)ethanol-to-hydroxytyrosol bioconversion ratio of 4-6% while the bioconversion ratio did not exceed 0.5% for reactions with control E. coli TOP10/pD1 cells. This experiment demonstrates that the hpaB- and hpaC-encoded aromatic monooxygenase HpaBC accepts 2-(3-hydroxyphenyl)ethanol as a substrate. This biotransformation of a substrate other than tyrosol to produce hydroxytyrosol had remained unprecedented so far.

### Example 4

### Improving the Bioconversion of Tyrosol to Hydroxytyrosol by Resting Escherichia coli Cells Expressing hpaB and hpaC Genes

To maximize the bioconversion yield of hydroxytyrosol from tyrosol, strategies were devised to increase cofactor availability by adding molecules such as glutathione or glycerol. In a typical experiment, a single colony of E. coli TOP10/pD1 was used to inoculate 50 mL of LB broth supplemented with ampicillin (100 mg/mL) for plasmid maintenance. The resulting culture was grown overnight at 37° C. with shaking at 250 rpm to ensure proper aeration. The overnight growth was used to inoculate several working cultures of 50 mL of LB broth supplemented with ampicillin to a starting O.D. at 600 nm of 0.1. The resulting cultures were shaken at 37° C. until an O.D. at 600 nm of 0.8-1.0 was reached, at which point IPTG was added to the medium to a final concentration of 0.5 mM. The cultures were further shaken at 37° C. for a 3.5 h induction period then shortly chilled on ice. The cells were harvested by centrifugation, washed with potassium phosphate buffer (50 mM, pH 7.0), harvested by centrifugation once more and finally resuspended in phosphate buffer (50 mM, pH 7.0) to a final O.D. at 600 nm of 20-30. Resulting cells were immediately set up in biotransformation reactions (5 mL) containing tyrosol (16 mM) in phosphate buffer (50 mM, pH 7.0). Reactions in which cells were added to reach an O.D. at 600 nm of 6-8 produced hydroxytyrosol in 23% conversion (mol/mol from tyrosol) after 18 h reaction time. Under the same reaction conditions but in the presence of glutathione (40 mM), hydroxytyrosol was produced in 49% conversion (mol/mol from tyrosol). Under similar reaction conditions but in the presence of glycerol (50 mM), hydroxytyrosol was produced in 62% conversion (mol/mol from tyrosol). When both glycerol (25 mM) and ascorbic acid (20 mM) were added to the reaction mixture, hydroxytyrosol conversion ratios increased to 83% (mol/mol from tyrosol). Under the same reaction conditions, 4-hydroxyphenylacetate (16 mM) was used instead of tyrosol as the starting material. In the presence of glutathione (50 mM) no expected 3,4-dihydroxyphenylacetate product was detected in the reaction mixture even after extended reaction times. When both ascorbate and glycerol were added, no more than 3% conversion into 3,4-dihydroxyphenylacetate (mol/mol from 4-hydroxyphenylacetate) was achieved, this being all the more surprising as 4-hydroxyphenylacetate is reported to be the natural substrate of HpaBC (Prieto M. A. et al. J. Bacteriol. (1993) 175:2162-2167).

### Example 5

### Bioconversion of Tyrosol to Hydroxytyrosol by Growing Escherichia coli Cells Expressing hpaB and hpaC Genes

To further test the robustness of hydroxytyrosol production from tyrosol, the HpaBC-catalyzed biotransformation was carried out using E. coli TOP/pD1 growing cells that express hpaB and hpaC genes. In a typical experiment, a single colony of E. coli TOP10/pD1 was used to inoculate 50 mL of LB broth supplemented with ampicillin (100 mg/mL) for plasmid maintenance. The resulting culture was grown overnight at 37° C. with shaking at 250 rpm to ensure proper aeration. The overnight growth was used to inoculate several working cultures of 50 mL of LB broth supplemented with ampicillin to a starting O.D. at 600 nm of 0.1. The resulting cultures were shaken at 37° C. until an O.D. at 600 nm of 0.8-1.0 was reached, at which point IPTG was added to the medium to a final concentration of 0.5 mM. Cultures were shaken at 37° C. and 250 rpm for another 4 h. Experiments were initiated (t=0) by addition of substrate tyrosol to a final concentration of 8.3 mM. Glycerol (27 mM) and ascorbic acid (20 mM) were also added to the culture medium at this point. Samples (1 mL) were withdrawn from growing E. coli TOP10/pD1 cultures at several time-points and the corresponding cell-free culture supernatants analyzed by HPLC. Typically, bacterial cultures were sampled just prior to substrate addition (t=−0.3 h) to provide a background check; immediately after substrate addition to provide an experimental measurement of initial substrate concentration (t=0); then 1-2 h after substrate addition to detect potential biosynthetic intermediates; and finally 16 h and 40 h after substrate addition to measure product and side-product concentrations. Growing E. coli TOP10/pD1 cells are able to transform tyrosol into hydroxytyrosol in 55-62% bioconversion ratio (mol/mol from tyrosol) within 1.6 h of reaction time. After 16 h of reaction, all tyrosol is consumed and converted into hydroxytyrosol in a 93-100% molar conversion ratio as judged by HPLC analysis.

### Examples of Hydroxytyrosol Production from Tyramine

### Example 6

### Construction of Plasmid pMPH

E. coli strain TOP10 (Invitrogen) was engineered to express genes encoding enzymatic activities that enable side-chain modification of tyramine via 4-hydroxyphenylaldehyde and via tyrosol to hydroxytyrosol.

The palR (SEQ ID NO:3) open reading frame (ORF) coding for phenylacetaldehyde reductase (SEQ ID NO:4) was amplified by PCR using Rhodococcus erythropolis (DSMZ 43297) chromosomal DNA as template, 5′-CCCGGGTAAGGAGGTGATCAAATGAAGGCAATCCAGTACACG-3′ (SmaI restriction site is underlined, ribosome binding site (rbs) and palR start codon are in boldface) as the forward primer, and 5′-GGATCCCTACAGACCAGGGACCACAACCG-3′ (BamHI restriction site is underlined) as the reverse primer. PCR mixtures (50 μL) contained 0.5 mg R. erythropolis (DSMZ 43297) chromosomal DNA, 50 pmol of each primer, 12.5 nmol of each deoxynucleotide (dNTPs), 5 U of Herculase DNA polymerase (Stratagene). PCR amplification started with a first denaturation step (95° C. for 5 min) followed by 35 repeats of temperature cycling steps (94° C. for 45 s, 55° C. for 45 s, and 72° C. for 90 s). The 1.1-kb PCR product was analyzed and gel-purified by agarose gel electrophoresis then mixed with vector pCR-XL-TOPO according to the TOPO® XL PCR Cloning Kit protocol (Invitrogen) to yield plasmid pPalR, which was subjected to DNA sequence analysis. The palR ORF was excised from plasmid pPalR by digestion with SmaI and BamHI and the 1.1-kb DNA fragment ligated to SmaI/BamHI-digested plasmid pD1 (also called pJFhpaBC) with T4 DNA ligase at 16° C. for 16 h. Ligation mixtures were used to transform E. coli TOP10 competent cells. Ampicillin-resistant transformants were selected on LB solid medium and analyzed for palR insertion, which afforded plasmid pJF palR hpaBC (also referred to as pPH).

The maoA ORF (SEQ ID NO:11) coding for monoamine oxidase (SEQ ID NO:12) was amplified by PCR using Escherichia coli MG1655 (CGSC # 7740) chromosomal DNA as template, 5′-GAATTCGGTACCTAAGGAGGTGATCAAATGGGAAGCCCCTCTCTG-3′ (EcoRI and KpnI restriction site are underlined, ribosome binding site (rbs) and maoA start codon are in boldface) as the forward primer, and 5′-CCCGGGTCACTTATCTTTCTTCAGCG-3′ (SmaI restriction site is underlined) as the reverse primer. PCR mixtures (50 μL) contained 0.5 mg E. coli MG1655 chromosomal DNA, 50 pmol of each primer, 12.5 nmol of each dNTPs, 5 U of Herculase DNA polymerase (Stratagene). PCR amplification started with a first denaturation step (95° C. for 5 min) followed by 35 repeats of temperature cycling steps (94° C. for 45 s, 55° C. for 45 s, and 72° C. for 150 s). The 2.3-kb PCR product was analyzed and gel-purified by agarose gel electrophoresis then mixed with vector pCR-XL-TOPO according to the TOPO® XL PCR Cloning Kit protocol (Invitrogen) to yield plasmid pMaoA, which was subjected to DNA sequence analysis. The maoA ORF was excised from plasmid pMaoA by digestion with EcoRI and SmaI and the 2.0-kb DNA fragment ligated to EcoRI/SmaI-digested plasmid pPH. Ligation mixtures were used to transform E. coli TOP10 competent cells. Ampicillin-resistant transformants were selected on LB solid medium and analyzed for maoA insertion, which afforded plasmid pJF maoA palR hpaBC (also referred to as pMPH).

### Example 7

### Bioconversion of Tyramine to Hydroxytyrosol by Growing Escherichia coli Cells Expressing maoA, palR, hpaB, and hpaC Genes

Inoculants were started by introducing either one single colony of E. coli TOP10/pMPH (picked off a freshly streaked agar plate) or 1 mL of E. coli TOP10/pMPH from a working cell bank (frozen in 20% glycerol) into 5 mL of M9 inoculation medium containing the appropriate antibiotic, in this case ampicillin (100 mg/L). Cultures were grown for 24 h. An aliquot of this culture was transferred to 50 mL of M9 induction medium containing ampicillin (100 mg/L), to a starting OD₆₀₀ of 0.025-0.05 (1% inoculum). The 50 mL culture was grown at 37° C. with agitation at 250 rpm to OD₆₀₀=0.5. Protein expression was then induced by addition of IPTG to a final concentration of 0.5 mM. The cultures were shaken at 37° C. and 250 rpm for another 2-3 h. Experiments were initiated (t=0) by addition of substrate tyramine to a final concentration of 2-3 mM. Samples (1 mL) were withdrawn from growing E. coli TOP10/pMPH cultures at several time-points and the corresponding cell-free culture supernatants analyzed by HPLC. Typically, bacterial cultures were sampled just prior to substrate addition (t=−0.3 h) to provide a background check; immediately after substrate addition to provide an experimental measurement of initial substrate concentration (t=0); then 1-2 h after substrate addition to detect potential biosynthetic intermediates; and finally 16 h after substrate addition to measure product and side-product concentrations (see Table 3). Growing E. coli TOP10/pMPH cells are able to transform tyramine into hydroxytyrosol in 82-93% bioconversion ratio (mol/mol from tyramine) within 16-22 h. Tyrosol, a predicted biosynthetic intermediate on the pathway from tyramine to hydroxytyrosol, could be transiently detected by HPLC analysis in the course of the biotransformation. Less than 4 mol % tyrosol remained in some cases at the end of the experiment. This leads to the conclusion that hydroxytyrosol can be produced from tyramine using a recombinant microorganism expressing an amine oxidase activity, an acetaldehyde reductase activity, and an aromatic hydroxylase activity.

TABLE 3
Evidence of hydroxytyrosol production from tyramine
catalyzed by growing E. coli strain TOP10/pMPH.

| | Time | Biomass | Concentrations in culture medium (mM)^{c} | | | Conversion |
|---|---|---|---|---|---|---|
| Entry^{a} | (h)^{b} | (OD₆₀₀) | Tyramine | Tyrosol | Hydroxytyrosol | (mol/mol)^{d} |
| 1.0^{e} | 0 | 1.9 | 2.72 | 0 | 0 | — |
| 1.1^{e} | 1 | 2.5 | 2.23 | 0.19 | 0.20 | — |
| 1.2^{e} | 16 | 3.5 | 0 | 0 | 2.23 | 82% |
| 2.0^{e} | 0 | 1.4 | 2.22 | 0 | 0 | — |
| 2.1^{e} | 1 | 2.8 | 2.03 | 0.23 | 0.14 | — |
| 2.2^{e} | 17 | 2.6 | 0 | 0 | 1.99 | 90% |
| 3.0^{f} | 0 | 1.8 | 1.93 | 0 | 0 | — |
| 3.1^{f} | 1.5 | 1.5 | 1.33 | 0.40 | 0.30 | — |
| 3.2^{f} | 22 | 3.7 | 0 | 0 | 1.73 | 90% |
| 4.0^{f} | 0 | 0.9 | 2.87 | 0 | 0 | — |
| 4.1^{f} | 1 | 1.3 | 2.35 | 0.35 | 0.07 | — |
| 4.2^{f} | 16 | 2.6 | 0 | 0.10 | 2.66 | 93% |

^{a}Entry series 1, 2, 3 and 4 correspond to several runs of the above-described experiment.
^{b}Time is counted starting from tyramine addition (t = 0).
^{c}As detected by HPLC analysis of cell-free culture supernatants.
^{d}Calculated as the molar ratio of final hydroxytyrosol to initial tyramine.
^{e}Experiment run in duplicate using E. coli strain TOP10/pMPH cells from a working cell bank (frozen in 20% glycerol).
^{f}Experiment run in duplicate starting from two different single colonies of E. coli strain TOP10/pMPH.

### Examples of Hydroxytyrosol Production from L-Tyrosine

### Example 8

### Construction of Plasmids

Enzymatic activities that decarboxylate L-tyrosine to yield tyramine are well-characterized in eukaryotic organisms, especially in plants, but to a lesser extent in prokaryotes. Microorganisms responsible for the occurrence of tyramine at potentially hazardous concentrations in fermented foods and beverages were identified as belonging to the genera Lactobacillus, Leuconostoc, Lactococcus, Enterococcus, or Carnobacterium and shown to express L-tyrosine decarboxylase activity. The functional role of putative L-tyrosine decarboxylase genes was recently established in a few bacteria such as Enterococcus faecalis (Connil N. et al. Appl. Environ. Microbiol. (2002) 68:3537-3544), Lactobacillus brevis IOEB 9809 (Lucas P. et al. FEMS Microbiol. Lett (2003) 229:65-71), and Carnobacterium divergens 508 (Coton M. et al. Food Microbiol. (2004) 21:125-130). A functional L-phenylalanine/L-tyrosine decarboxylase from Enterococcus faecium RM58 was also genetically characterized (Marcobal A. et al. FEMS Microbiol. Lett. (2006) 258:144-149). Putative L-tyrosine decarboxylase genes were identified by homology searches in all complete methanoarcheal genome sequences and even characterized in Methanocaldococcus jannaschii (Kezmarsky N. D. et al. Biochim. Biophys. Acta (2005) 1722:175-182).

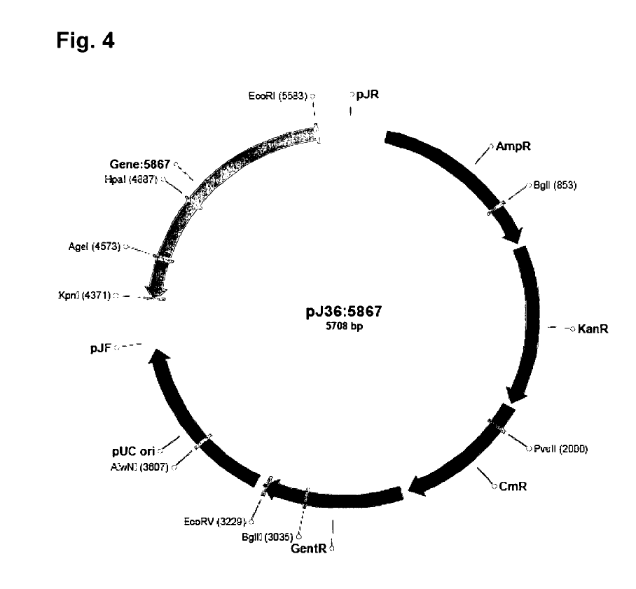

The tyrD ORF (SEQ ID NO:13) coding for L-tyrosine decarboxylase (SEQ ID NO:14) was made available by custom gene synthesis as carried out by DNA 2.0 Inc (USA) upon codon optimization of the mfnA gene from Methanocaldococcus jannaschii locus MJ0050 for improved heterologous protein expression in E. coli. The synthetic tyrD gene was received as an insert in plasmid pJ36:5867 (FIG. 4), from which it was excised by digestion with EcoRI and KpnI. The resulting 1.2-kb DNA fragment was ligated to EcoRI/KpnI-digested vector pUC18 to yield plasmid pUC tyrD (also referred to as pUCTD).

Digestion of plasmid pMPH with EcoRI and KpnI yielded two DNA fragments, 2.9-kb and 7.9-kb in size. The 1.2-kb tyrD locus was excised from plasmid pJ36:5867 by EcoRI and KpnI digestion and ligated to the gel-purified 7.9-kb DNA fragment from pMPH, yielding plasmid pJDΔMP in which maoA and palR genes are disrupted. The smaller 2.9-kb DNA fragment, also gel-purified from EcoRI/KpnI-digested plasmid pMPH, was ligated to KpnI-digested plasmid pJDΔMP to yield plasmid pJF tyrD maoA palR hpaBC (also referred to as pDMPH).

A gene coding for a putative L-tyrosine decarboxylase enzyme (SEQ ID NO:10) was identified in Pseudomonas putida KT2440 by searching publicly available databases for proteins homologous to known amino acid decarboxylase enzymes. The corresponding tyrDR ORF (SEQ ID NO:9) was amplified by PCR using P. putida KT2440 (DSMZ 6125) chromosomal DNA as template, 5′-GAATTCTAAGGAGGTGATCAAGTGACCCCCGAACAATTCCG-3′ (EcoRI restriction site is underlined, ribosome binding site (rbs) and tyrDR start codon are in boldface) as the forward primer, and 5′-GGTACCTCAGCCCTTGATCACGTCCTGC-3′ (KpnI restriction site is underlined) as the reverse primer. PCR mixtures (50 μL) contained 0.5 mg P. putida KT2440 chromosomal DNA, 50 μmol of each primer, 12.5 nmol of each dNTPs, 5 U of Herculase DNA polymerase (Stratagene). PCR amplification started with a first denaturation step (95° C. for 5 min) followed by 30 repeats of temperature cycling steps (94° C. for 60 s, 50° C. for 45 s, and 72° C. for 90 s). The 1.4-kb PCR product was analyzed and gel-purified by agarose gel electrophoresis then mixed with vector pCR-XL-TOPO according to the TOPO® XL PCR Cloning Kit (Invitrogen) to yield plasmid pTyrDR, which was subjected to DNA sequence analysis. The tyrDR ORF was excised from plasmid pTyrDR by digestion with EcoRI and KpnI and the 1.4-kb DNA fragment ligated to EcoRI/KpnI-digested vector pCK01. Ligation mixtures were used to transform E. coli TOP10 competent cells. Chloramphenicol-resistant transformants were selected on LB solid medium and analyzed for tyrDR insertion, which afforded plasmid pCKTyrDR.

### Example 9

### L-Phenylalanine/L-Tyrosine Decarboxylase Activity

E. coli TOP10 competent cells were transformed with high copy-number kanamycin-resistant pTyrDR and low copy-number chloramphenicol-resistant pCKTyrDR yielding E. coli strains TOP10/pTyrDR and TOP10/pCKTyrDR, respectively, which were tested for L-phenylalanine and L-tyrosine decarboxylating activity. In a typical procedure, inoculants were started by introducing one single colony of either E. coli strain TOP10/pTyrDR or E. coli strain TOP10/pCKTyrDR or E. coli strain TOP10 into 5 mL of LB medium containing the appropriate antibiotics. Cultures were grown overnight at 37° C. with agitation at 250 rpm and provided a 1% inoculum for 30 mL of fresh LB medium, supplemented with the appropriate antibiotics. The 30 mL cultures were grown at 37° C. with agitation at 250 rpm for 2 h then dispensed in 5 mL-aliquots into several culture tubes. The resulting 5 mL-cultures were treated with L-phenylalanine (5 mM), L-tyrosine (5 mM), or an equivalent volume of sterile water and incubated for 48 h at 37° C. with agitation at 250 rpm. Cells were removed by centrifugation. A 1 mL-sample of cell-free supernatant was treated with 1 mL disodium phosphate buffer (250 mM, pH 9.0), 0.1 mL of sodium hydroxide, and 2 mL of dansyl chloride solution (5 mg/mL in acetone), then vigorously mixed and incubated in the dark at 55° C. for 1 h to convert amines and residual amino acids into the corresponding fluorescent dansyl derivatives. Dansylated reaction components (10 μL) were separated by silica gel TLC using 1% triethanolamine in chloroform as the mobile phase. Fluorescent spots were compared with those of dansylated phenylethylamine and tyramine authentic samples. Both phenylethylamine and tyramine were detected in cell-free supernatants of biotransformation reactions involving tyrDR-expressing E. coli strains TOP10/pCKTyrDR and TOP10/pTyrDR. Higher concentrations of amines were detected when tyrDR was over-expressed using a high-copy plasmid (pTyrDR) versus a low-copy number plasmid (pCKTyrDR) clearly indicating that tyrDR encodes a functional L-phenylalanine/L-tyrosine decarboxylase, however with a preference for L-phenylalanine versus L-tyrosine as a substrate. A gene encoding a functional decarboxylase from non-pathogenic P. putida KT2440 able to convert L-phenylalanine and L-tyrosine into phenylethylamine and tyramine, respectively, was thus made available.

### Example 10

### L-Tyrosine Decarboxylase Activity

E. coli TOP10 competent cells were transformed with high copy-number ampicillin-resistant pUCTD yielding E. coli strain TOP10/pUCTD, which was tested for L-phenylalanine and L-tyrosine decarboxylating activity. In a typical procedure, inoculants were started by introducing one single colony of either E. coli strain TOP10/pUCTD or E. coli control strain TOP10 into 5 mL of LB medium containing the appropriate antibiotics. Cultures were grown overnight at 37° C. with agitation at 250 rpm and provided a 1% inoculum for 30 mL of fresh LB medium, supplemented with the appropriate antibiotics. The 30 mL cultures were grown at 37° C. with agitation at 250 rpm for 2 h then dispensed in 5 mL-aliquots into several culture tubes. The resulting 5 mL-cultures were treated with L-phenylalanine (5 mM), L-tyrosine (5 mM), or an equivalent volume of sterile water and incubated for 48 h at 37° C. with agitation at 250 rpm. Cells were removed by centrifugation. A 1 mL-sample of cell-free supernatant was treated with 1 mL disodium phosphate buffer (250 mM, pH 9.0), 0.1 mL of sodium hydroxide, and 2 mL of dansyl chloride solution (5 mg/mL in acetone), then vigorously mixed and incubated in the dark at 55° C. for 1 h to convert amines and residual amino acids into the corresponding fluorescent dansyl derivatives. Dansylated reaction components (10 μl) were separated by silica gel TLC using 1% triethanolamine in chloroform as the mobile phase. Fluorescent spots were compared with those of dansylated phenylethylamine and tyramine authentic samples. Tyramine was detected in cell-free supernatants of biotransformation reactions involving tyrD-expressing E. coli strain TOP10/pUCTD. No phenylethylamine was detected, confirming the specificity of the decarboxylase from M. jannaschii towards L-tyrosine. A synthetic gene encoding a functional decarboxylase of archaeal origin able to convert L-tyrosine into tyramine was thus made available.

### Example 11

### Bioconversion of L-Tyrosine to Hydroxytyrosol by E. coli TOP10/pDMPH Growing Cells in the Absence of Copper(II) Ions

Inoculants were started by introducing 1 mL of E. coli TOP10/pDMPH from a working cell bank (frozen in 20% glycerol) into 5 mL of M9 inoculation medium containing the appropriate antibiotic, in this case ampicillin (100 mg/L). Cultures were grown for 24 h. An aliquot of this culture was transferred to 50 mL of M9 induction medium containing ampicillin (100 mg/L), to a starting OD₆₀₀ of 0.025-0.05 (1% inoculum). The 50 mL culture was grown at 37° C. with agitation at 250 rpm to OD₆₀₀=0.5. Protein expression was then induced by addition of IPTG to a final concentration of 0.5 mM. The cultures were shaken at 37° C. and 250 rpm for another 2-3 h. Experiments were initiated (t=0) by addition of substrate L-tyrosine to final concentrations varying from 0.6 to 6 mM. Samples (1 mL) were withdrawn from growing E. coli TOP10/pDMPH cultures at several time-points and the corresponding cell-free culture supernatants analyzed by HPLC. Typically, bacterial cultures were sampled just prior to IPTG addition (t=−3.0 h) and just prior to substrate addition (t=−0.3 h) to provide background checks; immediately after substrate addition to provide an experimental measurement of initial substrate concentration (t=0); then 18 h and 42 h after substrate addition to measure product and side-product concentrations (see Table 4). E. coli TOP10/pDMPH growing cells successfully catalyzed the bioconversion of tyrosine to hydroxytyrosol regardless of the amount of initial tyrosine added at t=0 h. Good tyrosine-to-hydroxytyrosol bioconversion ratios ranging from 79-88% were achieved starting from tyrosine concentrations below 3.3 mM. Lower tyrosine-to-hydroxytyrosol bioconversion ratios ranging from 9-64% were reached when higher amounts of initial tyrosine ranging from 6-18 mM were added at t=0 h. This leads to the conclusion that hydroxytyrosol can be produced from tyrosine using a recombinant microorganism expressing genes that encode an amino acid decarboxylase activity, an amine oxidase activity, an acetaldehyde reductase activity, and an aromatic hydroxylase activity.

### Example 12

### Improvement of Hydroxytyrosol Biosynthesis by E. coli TOP10/pMPH and E. coli TOP10/pDMPH Growing Cells in the Presence of Copper(II) Ions

E. coli TOP10/pMPH and TOP10/pDMPH growing cells cultivated in M9 medium supplemented with casamino acids, which contain trace minerals such as copper ions (Nolan R. A. et al. App. Microbiol. (1972) 24:290-291), produce hydroxytyrosol in higher yields from substrates such as tyramine or tyrosine (≧2-3 mM) when treated with additional copper(II) ions. Copper(II) supplementation can take the form of, but is not limited to, addition of CuSO₄ or CuCl₂ aqueous solution to the bacterial culture. For optimal results, treatment with copper(II) should take place at the time of IPTG addition or at the time of substrate addition. In the absence of copper(II), E. coli TOP10/pMPH-catalyzed tyramine-to-hydroxytyrosol bioconversion and TOP10/pDMPH-catalyzed tyrosine-to-hydroxytyrosol bioconversion, do not cope well with initial substrate concentration higher than 2-3 mM, resulting in only partial conversion of the initial tyramine or tyrosine to tyrosol or hydroxytyrosol (see Table 4 and Table 5). In the presence of copper(II) ions, a marked increase in tyramine-to-hydroxytyrosol and tyrosine-to-hydroxytyrosol biotransformation ratios was demonstrated using growing bacterial cells of E. coli TOP10/pMPH and TOP10/pDMPH, respectively.

For example, E. coli TOP10/pMPH-catalyzed bioconversion of tyramine (5.6 mM) does not produce more than 1.2 mM hydroxytyrosol and 0.3 mM of tyrosol and leaves 4.6 mM tyramine untransformed after 42 h of reaction time in the absence of copper(II) ions. Under the same conditions, E. coli TOP10/pMPH growing cells treated with 50 μM CuSO₄ at the time of IPTG addition catalyze complete tyramine (5.1 mM) biotransformation within 18 h and produce up to 2.7 mM hydroxytyrosol and 0.4 mM tyrosol, in a calculated tyramine-to-hydroxytyrosol bioconversion ratio of 53% (mol/mol).

In another example, E. coli TOP10/pDMPH-catalyzed bioconversion of tyrosine (5.3 mM) stalled in the absence of copper(II): no residual tyrosine was detectable by HPLC analysis and 2.8 mM tyramine, 0.1 mM tyrosol, and 3.2 mM hydroxytyrosol had been produced within 18 h reaction time. In contrast, addition of 50 μM CuSO₄ to growing cultures of TOP10/pDMPH at the time of induction promoted excellent tyrosine-to-hydroxytyrosol bioconversion ratios. Up to 5.1 mM hydroxytyrosol was produced from 5.6 mM total starting substrates (5.4 mM tyrosine and 0.2 mM tyrosol) as detected by HPLC at t=0 h, resulting in a molar bioconversion ratio of 91% (mol/mol) in 18 h. Up to 7.8 mM hydroxytyrosol was produced from 10.1 mM starting substrates (9.9 mM tyrosine and 0.2 mM tyrosol) as detected by HPLC at t=0 h, resulting in a molar bioconversion ratio of 88% (mol/mol) in 18 h. Hydroxytyrosol was the only biotransformation product detected by HPLC 18 and 42 h after substrate addition. This example demonstrates that addition of copper(II) enhances hydroxytyrosol production by growing organisms such as E. coli TOP10/pMPH and E. coli TOP10/pDMPH, which express genes encoding HP- or FG-enzyme activities as described in the present invention.

TABLE 4
Evidence of hydroxytyrosol production from L-tyrosine catalyzed by
E. coli TOP10/pDMPH growing cells in the absence of copper(II) ions.

| | | | Concentrations in culture medium (mM)^{c} | | | | | |
|---|---|---|---|---|---|---|---|---|
| | Time | Biomass | | | | | Side | Conversion |
| Entry^{a} | (h)^{b} | (OD₆₀₀) | L-Tyrosine | Tyramine | Tyrosol | Hydroxytyrosol | Products^{d} | (mol/mol)^{e} |
| 1.1 | −3.0 | 0.62 | 0.84^{f} | 0.00 | 0.00 | 0.00 | 0.00 | — |
| 1.2 | −0.3 | 2.6 | 0.73 | 0.00 | 0.17 | 0.12 | 0.00 | — |
| 1.3 | 0 | 2.6 | 0.73 | 0.00 | 0.00 | 0.12 | 0.00 | — |
| 1.4 | 18 | 3.6 | 0.15 | 0.00 | 0.00 | 0.85 | 0.00 | — |
| 1.5 | 42 | 4.0 | 0.00 | 0.00 | 0.00 | 0.87 | 0.00 | 79% |
| 2.1 | −3.0 | 0.62 | 0.84^{f} | 0.00 | 0.00 | 0.00 | 0.00 | |
| 2.2 | −0.3 | 2.4 | 0.72 | 0.00 | 0.20 | 0.12 | 0.00 | |
| 2.3 | 0 | 2.4 | 1.81 | 0.00 | 0.20 | 0.11 | 0.03 | — |
| 2.4 | 18 | 3.4 | 0.15 | 0.00 | 0.00 | 1.81 | 0.00 | — |
| 2.5 | 42 | 3.5 | 0.00 | 0.00 | 0.00 | 1.88 | 0.00 | 87% |
| 3.1 | −3.0 | 0.62 | 0.84^{f} | 0.00 | 0.00 | 0.00 | 0.00 | — |
| 3.2 | −0.3 | 2.4 | 0.73 | 0.00 | 0.20 | 0.11 | 0.00 | — |
| 3.3 | 0 | 2.4 | 3.34 | 0.00 | 0.20 | 0.10 | 0.04 | — |
| 3.4 | 18 | 3.5 | 0.08 | 0.00 | 0.08 | 3.01 | 0.00 | — |
| 3.5 | 42 | 3.3 | 0.00 | 0.00 | 0.09 | 3.14 | 0.00 | 88% |
| 4.1 | −3.0 | 0.62 | 0.84^{f} | 0.00 | 0.00 | 0.00 | 0.00 | — |
| 4.2 | −0.3 | 2.4 | 0.73 | 0.00 | 0.18 | 0.12 | 0.00 | — |
| 4.3 | 0 | 2.4 | 6.07 | 0.00 | 0.18 | 0.11 | 0.01 | — |
| 4.4 | 18 | 3.7 | 0.17 | 2.79 | 0.09 | 3.61 | 0.12 | — |
| 4.5 | 42 | 3.6 | 0.00 | 2.59 | 0.35 | 3.99 | 0.33 | 64% |
| 5.1 | −3.0 | 0.62 | 0.84^{f} | 0.00 | 0.00 | 0.00 | 0.00 | — |
| 5.2 | −0.3 | 2.3 | 0.74 | 0.00 | 0.20 | 0.09 | 0.00 | — |
| 5.3 | 0 | 2.3 | 10.53 | 0.00 | 0.19 | 0.08 | 0.04 | — |
| 5.4 | 18 | 2.8 | 0.19 | 7.85 | 0.25 | 2.55 | 0.21 | — |
| 5.5 | 42 | 3.1 | 0.42 | 7.23 | 0.53 | 2.92 | 0.42 | 28% |
| 6.1 | −3.0 | 0.62 | 0.84^{f} | 0.00 | 0.00 | 0.00 | 0.00 | — |
| 6.2 | −0.3 | 2.4 | 0.73 | 0.00 | 0.19 | 0.12 | 0.00 | — |
| 6.3 | 0 | 2.4 | 18.31 | 0.00 | 0.17 | 0.06 | 0.00 | — |
| 6.4 | 18 | 4.1 | 3.94 | 6.62 | 0.92 | 1.41 | 0.63 | — |
| 6.5 | 42 | 5.0 | 3.78 | 7.91 | 1.13 | 1.36 | 1.17 | 9% |

^{a}Entry series 1, 2, 3, 4, 5, and 6 correspond to the above-described experiment using increasing L-tyrosine concentrations.
^{b}Time is counted starting from L-tyrosine addition (t = 0).
^{c}As detected by HPLC analysis of cell-free culture supernatants.
^{d}Sum of 4-hydroxyphenylacetic acid and 3,4-dihydroxyphenylacetic acid as detected by HPLC analysis of cell-free culture supernatants.
^{e}Calculated as the molar ratio of hydroxytyrosol produced to L-tyrosine consumed between t = 0 and t = 42 h; when applicable the contribution of tyrosol present at t = 0 h was excluded.
^{f}Before substrate addition L-tyrosine is present in the culture medium from casamino acids.

TABLE 5
Evidence of hydroxytyrosol production from L-tyrosine catalyzed by
E. coli TOP10/pDMPH growing cells in the presence of copper(II) ions.

| | | | Concentrations in culture medium (mM)^{c} | | | | | |
|---|---|---|---|---|---|---|---|---|
| | Time | Biomass | | | | | Side | Conversion |
| Entry^{a} | (h)^{b} | (OD₆₀₀) | L-Tyrosine | Tyramine | Tyrosol | Hydroxytyrosol | Products^{d} | (mol/mol)^{e} |
| 1.1 | −3.0 | 0.62 | 0.75^{f} | 0.00 | 0.00 | 0.00 | 0.00 | — |
| 1.2 | −0.3 | 2.6 | 0.76 | 0.00 | 0.18 | 0.10 | 0.00 | — |
| 1.3 | 0 | 2.6 | 0.76 | 0.00 | 0.18 | 0.11 | 0.00 | — |
| 1.4 | 18 | 3.6 | 0.00 | 0.00 | 0.00 | 0.85 | 0.00 | — |
| 1.5 | 42 | 4.0 | 0.00 | 0.00 | 0.00 | 0.84 | 0.00 | 72% |
| 2.1 | −3.0 | 0.62 | 0.75^{f} | 0.00 | 0.00 | 0.00 | 0.00 | |
| 2.2 | −0.3 | 2.4 | 0.76 | 0.00 | 0.19 | 0.11 | 0.00 | |
| 2.3 | 0 | 2.4 | 1.72 | 0.00 | 0.19 | 0.10 | 0.00 | — |
| 2.4 | 18 | 3.4 | 0.00 | 0.00 | 0.00 | 1.80 | 0.00 | — |
| 2.5 | 42 | 3.5 | 0.00 | 0.00 | 0.00 | 1.82 | 0.00 | 89% |
| 3.1 | −3.0 | 0.62 | 0.75^{f} | 0.00 | 0.00 | 0.00 | 0.00 | — |
| 3.2 | −0.3 | 2.4 | 0.76 | 0.00 | 0.19 | 0.11 | 0.00 | — |
| 3.3 | 0 | 2.4 | 3.24 | 0.00 | 0.20 | 0.11 | 0.00 | — |
| 3.4 | 18 | 3.5 | 0.00 | 0.00 | 0.00 | 3.25 | 0.00 | — |
| 3.5 | 42 | 3.3 | 0.00 | 0.00 | 0.00 | 3.34 | 0.00 | 94% |
| 4.1 | −3.0 | 0.62 | 0.75^{f} | 0.00 | 0.00 | 0.00 | 0.00 | — |
| 4.2 | −0.3 | 2.4 | 0.76 | 0.00 | 0.19 | 0.10 | 0.00 | — |
| 4.3 | 0 | 2.4 | 5.40 | 0.00 | 0.20 | 0.10 | 0.00 | — |
| 4.4 | 18 | 3.7 | 0.00 | 0.00 | 0.00 | 5.12 | 0.00 | — |
| 4.5 | 42 | 3.6 | 0.00 | 0.00 | 0.00 | 5.16 | 0.00 | 90% |
| 5.1 | −3.0 | 0.62 | 0.75^{f} | 0.00 | 0.00 | 0.00 | 0.00 | — |
| 5.2 | −0.3 | 2.3 | 0.76 | 0.00 | 0.18 | 0.10 | 0.00 | — |
| 5.3 | 0 | 2.3 | 9.93 | 0.00 | 0.20 | 0.09 | 0.00 | — |
| 5.4 | 18 | 2.8 | 0.51 | 0.00 | 0.26 | 7.78 | 0.00 | — |
| 5.5 | 42 | 3.1 | 0.00 | 0.00 | 0.31 | 7.90 | 0.00 | 79% |
| 6.1 | −3.0 | 0.62 | 0.75^{f} | 0.00 | 0.00 | 0.00 | 0.00 | — |
| 6.2 | −0.3 | 2.4 | 0.76 | 0.00 | 0.19 | 0.11 | 0.00 | — |
| 6.3 | 0 | 2.4 | 13.46 | 0.00 | 0.20 | 0.00 | 0.00 | — |
| 6.4 | 18 | 4.1 | 3.85 | 0.00 | 1.08 | 3.52 | 0.46 | — |
| 6.5 | 42 | 5.0 | 3.82 | 0.00 | 1.50 | 3.51 | 0.84 | 35% |

^{a}Entry series 1, 2, 3, 4, 5, and 6 correspond to the above-described experiment using increasing L-tyrosine concentrations.
^{b}Time is counted starting from L-tyrosine addition (t = 0).
^{c}As detected by HPLC analysis of cell-free culture supernatants.
^{d}Sum of 4-hydroxyphenylacetic acid and 3,4-dihydroxyphenylacetic acid as detected by HPLC analysis of cell-free culture supernatants.
^{e}Calculated as the molar ratio of hydroxytyrosol produced to L-tyrosine consumed between t = 0 and t = 42 h; when applicable the contribution of tyrosol present at t = 0 h was excluded.
^{f}Before substrate addition L-tyrosine is present in the culture medium from casamino acids.

Production of Hydroxytyrosol from Aromatic Substrates Other than Tyrosine, Tyramine, or Tyrosol

### Example 13

### Identification of Enzyme Activity and Encoding Gene to Transform Phenylpyruvate to Phenylacetaldehyde

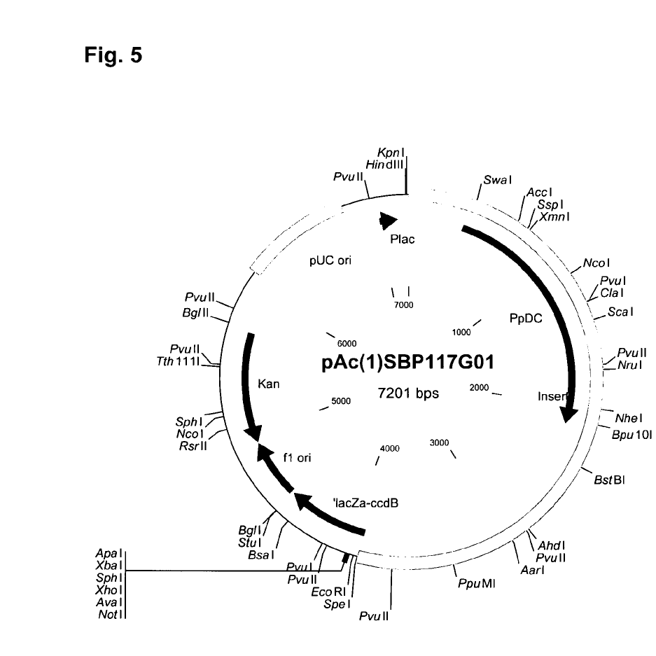

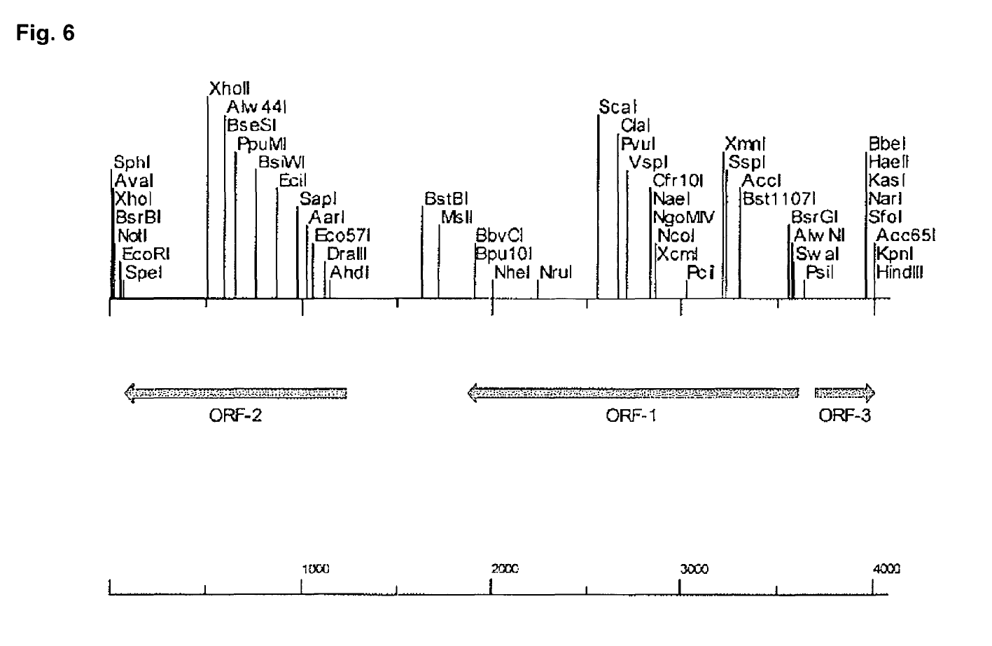

Appropriate enzymatic activities to transform phenylpyruvate to phenylacetaldehyde can be mainly found in eukaryotic organisms such as, for example, yeasts. To make available genes encoding such activity, sources of the appropriate enzymatic activity are preferable to be of bacterial origin to facilitate the engineering of microorganisms. A bacterium such as Acinetobacter calcoaceticus contains the appropriate enzymatic activity to transform phenylpyruvate to phenylacetaldehyde (Barrowman M. M. and Fewson C. A. Curr. Microbiol. (1985) 12:235-240). In order to make the gene encoding such activity available, chromosomal DNA from this bacterium was extracted and 50 μg partially digested with 2 U of the restriction endonuclease Sau3AI and the resulting mix of DNA fragments resolved in a preparative 0.6% agarose gel. The region of the gel containing DNA fragments of a size spanning 4-10 Kb was excised and DNA extracted from the gel matrix by the use of methodologies well know to those skilled in the art. The DNA fragments were finally dissolved in 20 μl of 10 mM Tris pH 8.6 μl of this DNA solution were utilized in a ligation reaction performed with 20 ng of BamHI digested pZErO™-2 vector (Invitrogen) using methodologies well know to those skilled in the art. After ligation was completed, the mixture was transformed in competent cells of E. coli DH10B and transformants were selected on LB agar plates containing kanamycin. This yielded more than 56,000 colonies which were pooled together and saved as glycerol stocks. Cells were spread on 2*TY agar plates containing 50 μg/ml kanamycin to obtain isolated colonies. Individual colonies were tested for their ability to transform phenylpyruvate. To do so, 96 well microtiter plates containing 0.2 ml of media 2*TY supplemented with 33 μg/ml kanamycin per well were inoculated with individual colonies. Colonies were allowed to develop into dense cultures by incubating the thus inoculated microtiter plates at 22° C. with shaking at 600 rpm for 48 h. After this time, a 150 μl sample from each well was transferred to a deepwell plate containing 140 μl of 120 mM phenylpyruvate in phosphate buffer (1 mM, pH 7.0), and incubated at 40° C. for 24 h. Samples from each well were then analyzed by ¹H-NMR spectroscopy. From the sample obtained from one of the wells, production of phenylacetate concomitant with consumption of phenylpyruvate could be identified. Plasmid DNA from the original colony (E. coli ACA117G1) showing such affect was extracted. This plasmid was labelled as pAc(1)SBP117g1, a map of this plasmid is represented in FIG. 5. The ca. 4 Kb fragment ligated to the vector backbone was sequenced. This DNA sequence is identified as SEQ ID NO:15. This DNA sequence was analyzed by the use of DNA analysis tools based in computer software well known to those skilled in the art. A representative sequence map of this sequence is represented in FIG. 6. A section of this DNA sequence included a potential open reading frame which encoded a protein sequence that was predicted by DNA software analysis to present homology with diverse decarboxylase enzymes. The DNA sequence of this open reading frame (orf) is described as SEQ ID NO:16. The protein sequence encoded by this DNA sequence is identified as SEQ ID NO:17. Although phenylacetaldehyde could not be detected, the production of phenylacetate from phenylpyruvate is an indication of phenylacetaldehyde formation as known in publicly available literature (Asakawa T. et al. Biochim. Biophys. Acta. (1968) 170:375-391). Therefore, the sequence of the gene encoding an enzymatic activity capable of transforming phenylpyruvate to phenylacetaldehyde was this way made available. Any person skilled in the art will recognize that such pyruvate decarboxylase activity is also capable of transforming 4-hydroxyphenylpyruvate to 4-hydroxyphenylacetaldehyde.

### Example 14

### Production of Hydroxytyrosol from Dopamine by Recombinant E. coli Strains Expressing Genes Encoding Amine Oxidase and Aldehyde Reductase Enzymatic Activities

Inoculants were started by introducing 1 mL of a suspension of E. coli TOP10/pMPH cells in 20% glycerol from a working cell bank into 5 mL of M9 inoculation medium containing the appropriate antibiotic, in this case ampicillin (100 mg/L). Cultures were grown for 24 h. An aliquot of this culture was transferred to 50 mL of M9 induction medium containing ampicillin (100 mg/L), to a starting OD₆₀₀₀ of 0.025-0.05 (1% inoculum). The 50 mL culture was grown at 37° C. with agitation at 250 rpm to OD₆₀₀≈0.5. Protein expression was then induced by adding IPTG to a final concentration of 0.5 mM. Cultures were shaken at 37° C. and 250 rpm for ˜3 h then treated with dopamine to an initial concentration of ˜1.6 mM as measured by HPLC at t=0 h. Dopamine-treated E. coli TOP10/pMPH growing cultures expressing maoA, palR, and hpaBC genes were assayed for hydroxytyrosol production. Control experiments were set up in parallel following the same experimental protocol, in which E. coli TOP10/pD1 growing cells expressing hpaBC genes were treated with dopamine (2-(3,4-dihydroxyphenyl)ethylamine). Up to ˜1.3 mM hydroxytyrosol was detected by HPLC analysis of cell-free supernatants of E. coli TOP10/pMPH cultures 18 h after substrate addition, which amounts to a dopamine-to-hydroxytyrosol bioconversion ratio of ˜81% (mol/mol). Hydroxytyrosol titers remained stable as judged by HPLC analysis of culture supernatants 42 h after substrate addition. No hydroxytyrosol was detected in cell-free supernatants of dopamine-treated E. coli TOP10/pD1 control cultures, which is consistent with monoamine oxidase activity (encoded by the maoA gene) and phenylacetaldehyde reductase (encoded by the palR gene) catalyzing the two-step bioconversion of dopamine to hydroxytyrosol. Some 3,4-dihydroxyphenylacetic acid (˜0.4 mM) was detected by HPLC as a minor side product in culture supernatants. The known existence of phenylacetaldehyde dehydrogenase activity (PAD) in E. coli K-12 (Parrott et al. J. Gen. Microbiol. (1987) 133:347-351; Hanlon et al. Microbiol. (1997) 143:513-518) accounts for 3,4-dihydroxyphenylacetic acid production from 3,4-dihydroxyphenylacetaldehyde, which is the biosynthetic intermediate formed upon MaoA-catalyzed oxidative deamination of dopamine. Our results provide strong evidence that the enzymatic activities encoded by genes such as maoA and palR expressed by growing E. coli TOP10/pMPH cells lead to bioconversion of dopamine to hydroxytyrosol via the intermediacy of 3,4-dihydroxyphenylacetaldehyde. Enzymatic activities encoded by genes such as maoA and palR allow for the modification of the ethylamine side-chain of dopamine and its conversion into the ethylalcohol side-chain of hydroxytyrosol.

### Example 15

### Production of 2-phenylethanol from 2-phenylethylamine by Recombinant E. coli Strains Expressing Genes Encoding Amine Oxidase and Aldehyde Reductase Enzymatic Activities and Production of Hydroxytyrosol from 2-phenylethanol

E. coli strain TOP10/pMPH was cultivated in 50 mL M9 induction medium and induced for gene expression using IPTG as described in the previous examples. After ˜3 h shaking at 37° C. and 250 rpm, cultures were treated with phenylethylamine to an initial concentration of ˜2.2 mM as measured by HPLC at t=0 h. Phenylethylamine-treated E. coli TOP10/pMPH growing cultures expressing maoA, palR, and hpaBC genes were assayed for metabolites production. Control experiments were set up in parallel following the same experimental protocol, in which E. coli TOP10/pD1 growing cells expressing hpaBC genes were treated with phenylethylamine. Up to ˜1.5 mM phenylethanol was detected by HPLC analysis of cell-free supernatants of E. coli TOP10/pMPH cultures 42 h after substrate addition, which amounts to a phenylethylamine-to-phenylethanol bioconversion ratio of ˜68% (mol/mol). No phenylethanol was detected in cell-free supernatants of phenylethylamine-treated E. coli TOP10/pD1 control cultures, which is consistent with monoamine oxidase activity (encoded by the maoA gene) and phenylacetaldehyde reductase (encoded by the palR gene) catalyzing the two-step bioconversion of phenylethylamine to phenylethanol. Enzymatic activities encoded by genes such as maoA and palR allow for the modification of the ethylamine side-chain of phenylethylamine and its conversion into the ethylalcohol side-chain of phenylethanol. Further elaboration of phenylethanol to hydroxytyrosol should be possible using hydroxylating enzymes such as toluene monooxygenases. For example, toluene para-monooxygenase (TpMO) from Ralstonia pickettii PKO1 (Fishman et al. J. Biol. Chem. (2005) 280:506-514) and toluene 4-monooxygenase (T4MO) from Pseudomonas mendocina KR1 (Pikus et al. Biochemistry (1997) 36:9283-9289) should catalyze the hydroxylation of phenylethanol into tyrosol or 2-(3-hydroxyphenyl)ethanol or a mixture of both 3- and 4-hydroxyphenylethanol derivatives. T4MO was reported to catalyze hydroxylation of ethylbenzene. Both enzymes are multi-component non-heme diiron monooxygenases encoded by six genes and comprising a hydroxylase component structured in three alpha- (SEQ ID NO:19 and 25), beta-(SEQ ID NO:21 and 27), and gamma-(SEQ ID NO:23 and 29) subunits. The regioselectivity of toluene monooxygenase-catalyzed hydroxylation can be modified by mutation of the gene encoding the alpha-hydroxylase subunit (SEQ ID NO:18 and 24). Any person skilled in the art will recognize that either naturally occurring or mutant enzymes of the toluene monooxygenase family should be amenable to carry out the hydroxylation of phenylethanol at the para- or meta-position to yield substrates such as tyrosol or 2-(3-hydroxyphenyl)ethanol, respectively, that can be further elaborated into hydroxytyrosol using the invention described herein.

### Example 16

### Production of Hydroxytyrosol from L-Phenylalanine Via 2-phenylethanol by Recombinant E. coli Strains Expressing Genes Encoding Amino Acid Decarboxylase, Amine Oxidase, Aldehyde Reductase Activities, Toluene Monooxygenase Activities, and Tyrosol Hydroxylase

Any person skilled in the art will recognize that hydroxytyrosol can be produced from L-phenylalanine by combining enzymatic activities made available in the present invention. L-Phenylalanine can be converted into 2-phenylethanol by combining the above-described tyrDR gene encoding L-phenylalanine/L-tyrosine decarboxylase activity with the maoA and palR genes encoding amine oxidase and aldehyde reductase activities, respectively. The resulting 2-phenylethanol can be further elaborated into tyrosol, or 2-(3-hydroxyphenyl)ethanol, or hydroxytyrosol, or a mixture thereof, by introducing a hydroxyl group at the para- and/or meta-positions using enzymes such as toluene 4-monooxygenase T4MO (SEQ ID NO:25, SEQ ID NO:27, and SEQ ID NO:29) or toluene para-monooxygenase TpMO (SEQ ID NO:19, SEQ ID NO:21, and SEQ ID NO:23) encoded by genes such as tmoAEB (SEQ ID NO:24, SEQ ID NO:26, and SEQ ID NO:28) or tbuA1A2U (SEQ ID NO:18, SEQ ID NO:20, and SEQ ID NO:22), respectively. Both tyrosol and 2-(3-hydroxyphenyl)ethanol can be further hydroxylated to yield hydroxytyrosol using a hydroxylating enzyme such as HpaBC.

### Example 17

### Production of Hydroxytyrosol from L-Phenylalanine Via L-Tyrosine by Recombinant E. coli Strains Expressing Genes Encoding L-Phenylalanine 4-Monooxygenase, Amino Acid Decarboxylase, Amine Oxidase, Aldehyde Reductase, and Hydroxylase Activities

A person skilled in the art will recognize that hydroxytyrosol can also be produced from L-phenylalanine by combining the phhAB genes (SEQ ID NO:30 and SEQ ID NO:32 or SEQ ID NO:34 and SEQ ID NO:36) encoding L-phenylalanine 4-monooxygenase PhhAB (SEQ ID NO:31 and SEQ ID NO:33, respectively, or SEQ ID NO:35 and SEQ ID NO:37, respectively) that catalyzes the conversion of L-phenylalanine to L-tyrosine, with the tyrD, maoA, palR, and hpaBC genes encoding enzyme activities that allow the bioconversion of L-tyrosine to hydroxytyrosol. L-Phenylalanine 4-monooxygenase genes can be made available from Pseudomonas aeruginosa (Zhao et al. Proc. Natl. Acad. Sci. USA (1994) 91:1366-1370) or Pseudomonas putida (Carmen Herrera & Ramos J. Mol. Biol. (2007) 366:1374-1386) genomic DNA by using techniques well know to any person skilled in the art.

Hydroxytyrosol can also be produced from L-phenylalanine using a combination of the phhAB, tyrD, maoA, palR, genes and a gene encoding tyrosinase activity that catalyzes the conversion of phenolic substrates such as tyrosol or L-tyrosine to the corresponding catechols such as hydroxytyrosol and L-3,4-dihydroxyphenylalanine (L-dopa), respectively. The amino acid L-dopa can be further processed into hydroxytyrosol using enzyme activities encoded by genes described herein such as tyrD and tyrDR for the decarboxylation step, maoA for the oxidative deamination step, and palR for the reduction step. Tyrosinase genes (SEQ ID NO:1 or SEQ ID NO:38 or SEQ ID NO:40) encoding an HP-enzymes (SEQ ID NO:2 or SEQ ID NO:39 or SEQ ID NO:41, respectively) are ubiquitous and can be made available from the mushroom Agaricus bisporus (Wichers al. Appl. Microbiol. Biotechnol. (2003) 61:336-341) or the fungus Pycnoporus sanguineus (Halaouli et al. Appl. Microbiol. Biotechnol. (2006) 70:580-589) by using techniques well know to those skilled in the art.

1. Use of a polynucleotide encoding an enzyme involved in the catabolism of aromatic compounds for the production of hydroxytyrosol, wherein said enzyme is involved in the design of the hydroxytyrosol specific hydroxylation pattern (HP protein) or in the design of the hydroxytyrosol specific functional group (FG protein); and wherein said polynucleotide is selected from the group consisting of:
a) polynucleotides encoding a protein comprising the amino acid sequence according to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, or SEQ ID NO: 41;
b) polynucleotides comprising the nucleotide sequence according to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40;
c) polynucleotides encoding a fragment or derivative of a polypeptide encoded by a polynucleotide of any of (a) or (b) wherein in said derivative one or more amino acid residues are conservatively substituted compared to said polypeptide, and said fragment or derivative has the activity of a HP or FG protein;
d) polynucleotides the complementary strand of which hybridizes under stringent conditions to a polynucleotide as defined in any one of (a) to (c) and which encode a HP or FG protein;
e) polynucleotides which are at least 90% identical to a polynucleotide as defined in any one of (a) to (d) and which encode a HP or FG polypeptide; and
f) the complementary strand of a polynucleotide as defined in (a) to (e).

2. A vector containing at least one polynucleotide according to claim 1.

3. The vector of claim 2 in which the polynucleotide is operatively linked to expression control sequences allowing the expression in prokaryotic or eukaryotic host cells.

4. A polypeptide which has the activity of a HP or FG protein and which is selected from the group consisting of:
a) polypeptides as shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO:17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, or SEQ ID NO: 41;
b) polypeptides comprising an amino acid sequence comprising a fragment or derivative of a polypeptide sequence according to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, or SEQ ID NO: 41;
c) polypeptides comprising an amino acid sequence encoded by a fragment or derivative of a polynucleotide sequence according to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40; and
d) polypeptides which are at least identical to a polypeptide according to (a) to (c) and which have the activity of a HP or FG polypeptide.

5. A microorganism capable of the production of hydroxytyrosol, characterized in that it expresses at least one polynucleotide encoding an enzyme involved in the catabolism of aromatic compounds, wherein said polynucleotide is selected from the group consisting of:
a) polynucleotides encoding a protein comprising the amino acid sequence according to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, or SEQ ID NO: 41;
b) polynucleotides comprising the nucleotide sequence according to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40;
c) polynucleotides encoding a fragment or derivative of a polypeptide encoded by a polynucleotide of any of (a) or (b) wherein in said derivative one or more amino acid residues are conservatively substituted compared to said polypeptide, and said fragment or derivative has the activity of a HP or FG protein;
d) polynucleotides the complementary strand of which hybridizes under stringent conditions to a polynucleotide as defined in any one of (a) to (c) and which encode a HP or FG protein;
e) polynucleotides which are at least 90% identical to a polynucleotide as defined in any one of (a) to (d) and which encode a HP or FG polypeptide; and
f) the complementary strand of a polynucleotide as defined in (a) to (e).

6. A genetically engineered microorganism capable of the production of hydroxytyrosol, characterized in that it has been transformed or transfected by at least one polynucleotide encoding an enzyme involved in the catabolism of aromatic compounds, wherein said polynucleotide is selected from the group consisting of:
a) polynucleotides encoding a protein comprising the amino acid sequence according to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO; 33, SEQ ID NO: 35, SEQ ID NO: 37, SEQ ID NO: 39, or SEQ ID NO: 41;
b) polynucleotides comprising the nucleotide sequence according to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40;
c) polynucleotides encoding a fragment or derivative of a polypeptide encoded by a polynucleotide of any of (a) or (b) wherein in said derivative one or more amino acid residues are conservatively substituted compared to said polypeptide, and said fragment or derivative has the activity of a HP or FG protein;
d) polynucleotides the complementary strand of which hybridizes under stringent conditions to a polynucleotide as defined in any one of (a) to (c) and which encode a HP or FG protein;
e) polynucleotides which are at least 90% identical to a polynucleotide as defined in any one of (a) to (d) and which encode a HP or FG polypeptide; and
f) the complementary strand of a polynucleotide as defined in (a) to (e).

7. The microorganism according to claim 5, characterized in that it expresses or has been transformed or transfected by at least two polynucleotides.

8. The microorganism according to claim 5, characterized in that it expresses or has been transformed or transfected by at least three polynucleotides.

9. The microorganism according to claim 5, characterized in that it expresses or has been transformed or transfected by at least four polynucleotides.

10. The microorganism according to claim 5, characterized in that it expresses or has been transformed or transfected by at least five polynucleotides.

11. A microorganism genetically altered by at least one polynucleotide to encode a protein selected from the group consisting of enzymes which are capable of transforming L-phenylalanine to phenylpyruvate, phenylpyruvate to phenylacetaldehyde, phenylacetaldehyde to phenylethanol, phenylethanol to Hy-T, L-phenylalanine to phenylethylamine, phenylethylamine to phenylacetaldehyde, phenylethanol to tyrosol, tyrosol to Hy-T, L-tyrosine to 4-hydroxyphenylpyruvate, 4-hydroxyphenylpyruvate to 4-hydroxyphenylacetaldehyde, 4-hydroxyphenylacetaldehyde to tyrosol, L-tyrosine to tyramine, tyramine to 4-hydroxyphenylacetaldehyde, prephenate to L-tyrosine, prephenate to L-phenylalanine, prephenate to 4-hydroxyphenylpyruvate, prephenate to phenylpyruvate, L-phenylalanine to L-tyrosine, phenylethylamine to tyramine, phenylacetaldehyde to 4-hydroxyphenylacetaldehyde, L-tyrosine to L-dopa, L-dopa to dopamine, dopamine to 3,4-dihydroxyphenylacetaldehyde, and 3,4-dihydroxyphenylacetaldehyde to Hy-T.

12. The microorganism according to claim 5, which is not pathogenic.

13. A process for producing cells capable of expressing at least one polypeptide, comprising genetically engineering cells with the polynucleotide(s) according to claim 1 or with a vector containing at least the polynucleotide(s).

14. The process for the direct production of Hy-T, wherein a microorganism according to claim 5 is cultivated in a aqueous nutrient medium under conditions that allow the direct production of Hy-T and wherein Hy-T is isolated as the fermentation product.

15. The process according to claim 14, characterized in that glutathione and/or glycerol and/or ascorbic acid is added to the reaction medium.

16. The process according to claim 14, characterized in that a copper(II) salt is added to the reaction medium.

17. The process according to claim 14, wherein Hy-T is produced by resting cells.

18. The process according to claim 14, wherein Hy-T is produced by growing cells.

19. The process according to claim 14, wherein Hy-T is produced by a non-pathogenic organism.


SEQUENCE LISTING (41 sequences)

SEQ ID NO: 1 - DNA, 1857 bp, Pycnoporus sanguineus BRFM49
atgtcacacttcatcgttactgggcctgtaggaggtcagactgagggcgctcctgctcccaaccgcctcgaaatcaacgacttcgtcaagaatgaggagttcttctcgctttacgtccaggctctcgatatcatgtatggactgaagcaggaggaactgatctcgttcttccagatcggtggcattcatggattgccatacgttgcctggagtgatgccggagcggatgaccctgctgagccgtccgggtactgtacccatggctccgtactgttcccgacctggcataggccttacgtcgcactatatgagcaaatcttgcacaagtatgctggagagatcgctgataagtacacggtcgacaaaccgcgttggcagaaggcagcggccgacctgcgccaacccttctgggactgggccaagaacacgctgcctcctcctgaagtcatctctctcgacaaagtcacgattacgacaccagatggacagaggacgcaagttgacaatccactccgtcgctaccgcttccatccgatcgaccccagcttcccagagccatacagcaactggccagcgacactgagacatccgacaagtgatggctcggatgccaaagacaacgtgaaggatctcactactactctgaaggcggaccagcctgatatcacgacgaagacgtataatctattgaccagagtgcacacgtggccggcgttcagcaaccacactccaggcgatggcggcagctccagtaacagtcttgaggccattcacgaccacatccatgactcagttggcggcggaggccagatgggagacccgtccgtggcaggcttcgacccaatcttcttcctgcaccattgccaagttgatcgtcttcttgcactgtggtccgccttgaaccccggcgtgtgggtcaacagctctagctccgaagatggcacctacacgatcccgcctgactctaccgtggaccaaactactgcattgacgcccttctgggatacccaaagcacattctggacgtccttccagtctgctggagtctcgcccagccaatttggctattcttaccccgagtttaacggtctcaacctgcaagatcagaaggctgtgaaagatcacatcgccgaggtcgtgaacgagctctacggtcatcgcatgcggaaaaccttccctttcccccagctccaggcagtttccgtagccaagcagggcgacgccgtcactccatccgtggctaccgattcagtgtcgtcttctaccacacctgccgaaaatcccgcatcccgcgaggatgcctctgataaggacacagagccgacgctcaatgtagaggttgccgcgccaggcgcgcacttgacctccaccaagtattgggactggactgctcgcattcatgtcaagaagtacgaagtcggaggcagcttcagcgtcctgctcttcctgggtgcaatccccgagaacccagcggattggcgcacgagccccaactacgttggcggtcatcatgctttcgtgaatagctcaccgcagcgctgcgctaactgccgtggtcaaggcgaccttgtcatcgaaggcttcgtccatctcaacgaggcgatcgcccgccatgcgcacctcgactccttcgatccaaccgtcgtgaggccgtacctcacgcgcgagttgcactggggtgtgatgaaggtgaatggcaccgtcgtgcccctgcaagacgtcccgtcgctcgaggttgtcgtcctctcaactcctcttacccttcctccgggagagccattccctgtccccggaacgcccgtcaatcatcatgacatcacccatggacgtcctggtggctctcaccacacgcactaa

SEQ ID NO: 2 - protein, 618 aa, Pycnoporus sanguineus BRFM49
MSHFIVTGPVGGQTEGAPAPNRLEINDFVKNEEFFSLYVQALDIMYGLKQEELISFFQIGGIHGLPYVAWSDAGADDPAEPSGYCTHGSVLFPTWHRPYVALYEQILHKYAGEIADKYTVDKPRWQKAAADLRQPFWDWAKNTLPPPEVISLDKVTITTPDGQRTQVDNPLRRYRFHPIDPSFPEPYSNWPATLRHPTSDGSDAKDNVKDLTTTLKADQPDITTKTYNLLTRVHTWPAFSNHTPGDGGSSSNSLEAIHDHIHDSVGGGGQMGDPSVAGFDPIFFLHHCQVDRLLALWSALNPGVWVNSSSSEDGTYTIPPDSTVDQTTALTPFWDTQSTFWTSFQSAGVSPSQFGYSYPEFNGLNLQDQKAVKDHIAEVVNELYGHRMRKTFPFPQLQAVSVAKQGDAVTPSVATDSVSSSTTPAENPASREDASDKDTEPTLNVEVAAPGAHLTSTKYWDWTARIHVKKYEVGGSFSVLLFLGAIPENPADWRTSPNYVGGHHAFVNSSPQRCANCRGQGDLVIEGFVHLNEAIARHAHLDSFDPTVVRPYLTRELHWGVMKVNGTVVPLQDVPSLEVVVLSTPLTLPPGEPFPVPGTPVNHHDITHGRPGGSHHTH

SEQ ID NO: 3 - DNA, 1047 bp, Rhodococcus erythropolis
atgaaggcaatccagtacacgagaatcggcgcggaacccgaactcacggagattcccaaacccgagcccggtccaggtgaagtgctcctggaagtcaccgctgccggcgtctgccactcggacgacttcatcatgagcctgcccgaagagcagtacacctacggccttccgctcacgctcggccacgaaggcgcaggcaaggtcgccgccgtcggcgagggtgtcgaaggtctcgacatcggaaccaatgtcgtcgtctacgggccttggggttgtggcaactgttggcactgctcacaaggactcgagaactattgctctcgcgcccaagaactcggaatcaatcctcccggtctcggtgcacccggcgcgttggccgagttcatgatcgtcgattctcctcgccaccttgtcccgatcggtgacctcgacccggtcaagacggtgccgctgaccgacgccggtctgacgccgtatcacgcgatcaagcgttctctgccgaaacttcgcggaggctcgtacgcggttgtcattggtaccggcgggctcggccacgtcgccattcagctcctccgtcacctctcggcggcaacggtcatcgctttggacgtgagcgcggacaagctcgaactggcaaccaaggtaggcgctcacgaagtggttctgtccgacaaggacgcggccgagaacgtccgcaagatcactggaagtcaaggcgccgcactggttctcgacttcgtcggctaccagcccaccatcgacaccgcgatggctgtcgccggcgtcggatcagacgtcacgatcgtcgggatcggggacggccaggcccacgccaaagtcgggttcttccaaagtccttacgaggcttcggtgacagttccgtattggggtgcccgcaacgagttgatcgaattgatcgacctcgcccacgccggcatcttcgacatcgcggtggagaccttcagtctcgacaacggtgccgaagcgtatcgacgactggctgccggaacgctaagcggccgtgcggttgtggtccctggtctgtag

SEQ ID NO: 4 - protein, 348 aa, Rhodococcus erythropolis
MKAIQYTRIGAEPELTEIPKPEPGPGEVLLEVTAAGVCHSDDFIMSLPEEQYTYGLPLTLGHEGAGKVAAVGEGVEGLDIGTNVVVYGPWGCGNCWHCSQGLENYCSRAQELGINPPGLGAPGALAEFMIVDSPRHLVPIGDLDPVKTVPLTDAGLTPYHAIKRSLPKLRGGSYAVVIGTGGLGHVAIQLLRHLSAATVIALDVSADKLELATKVGAHEVVLSDKDAAENVRKITGSQGAALVLDFVGYQPTIDTAMAVAGVGSDVTIVGIGDGQAHAKVGFFQSPYEASVTVPYWGARNELIELIDLAHAGIFDIAVETFSLDNGAEAYRRLAAGTLSGRAVVVPGL

SEQ ID NO: 5 - DNA, 1563 bp, Escherichia coli
atgaaaccagaagatttccgcgccagtacccaacgtccgttcaccggggaagagtatctgaaaagcctgcaggatggtcgcgagatctatatctatggcgagcgagtgaaagacgtcactactcatccggcatttcgtaatgcggctgcgtctgttgcccaactgtacgacgcgctacacaaaccggagatgcaggactctctgtgctggaacaccgacaccggcagcggcggctatacccataaattcttccgcgtggcgaaaagtgccgacgacctgcgccacgaacgcgatgccatcgctgagtggtcacgcctgagctatggctggatgggccgtaccccagactacaaagctgctttcggttgcgcactgggcggaactccgggcttttacggtcagttcgagcagaacgcccgtaactggtacacccgtattcaggaaactggcctctactttaaccacgcgattgttaacccaccgatcgatcgtcatttgccgaccgataaagtaaaagacgtttacatcaagctggaaaaagagactgacgccgggattatcgtcagcggtgcgaaagtggttgccaccaactcggcgctgactcactacaacatgattggcttcggctcggcacaagtaatgggcgaaaacccggacttcgcactgatgttcgttgcgccaatggatgccgatggcgtcaaattaatctcccgcgcctcttatgagatggtcgcgggtgctaccggctcaccgtatgactacccgctctccagccgcttcgatgagaacgatgcgattctggtgatggataacgtgctgatcccatgggaaaacgtgctgctctaccgcgattttgatcgctgccgtcgctggacgatggaaggcggtttcgcccgtatgtatccgctgcaagcctgtgtgcgcctggcagtgaaactcgacttcattacggcactgctgaaaaaatcactcgaatgtaccggcaccctggagttccgtggtgtgcaggccgatctcggtgaagtggtggcgtggcgcaacaccttctgggcattgagtgactcgatgtgttctgaagcgacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaacaatccgcagatcgaccagtatctggcgaagtatgtgcgcggttcgaacggtatggatcacgtccagcgcatcaagatcctcaaactgatgtgggatgccattggcagcgagtttggtggtcgtcacgaactgtatgaaatcaactactccggtagccaggatgagattcgcctgcagtgtctgcgccaggcacaaagctccggcaatatggacaagatgatggcgatggttgatcgctgcctgtcggaatacgaccagaacggctggactgtgccgcacctgcacaacaacgacgatatcaacatgctggataagctgctgaaataa

SEQ ID NO: 6 - protein, 520 aa, Escherichia coli
MKPEDFRASTQRPFTGEEYLKSLQDGREIYIYGERVKDVTTHPAFRNAAASVAQLYDALHKPEMQDSLCWNTDTGSGGYTHKFFRVAKSADDLRHERDAIAEWSRLSYGWMGRTPDYKAAFGCALGGTPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLLYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQSSGNMDKMMAMVDRCLSEYDQNGWTVPHLHNNDDINMLDKLLK

SEQ ID NO: 7 - DNA, 513 bp, Escherichia coli
atgcaattagatgaacaacgcctgcgctttcgtgacgcaatggccagcctgtcggcagcggtaaatattatcaccaccgagggcgacgccggacaatgcgggattacggcaacggccgtctgctcggtcacggatacaccaccatcgctgatggtgtgcattaacgccaacagtgcgatgaacccggtttttcagggcaacggtaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcacggacttatctactttaaacgccgtttccatccggtgatgctggaaatggaagctgcgatttaa

SEQ ID NO: 8 - protein, 170 aa, Escherichia coli
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 9 - DNA, 1413 bp, Pseudomonas putida
gtgacccccgaacaattccgccagtacggccaccaactgatcgacctgattgccgactaccgccagaccgtgggcgaacgcccggtcatggcccaggtcgaacctggctatctcaaggccgccttgcccgcaactgcccctcaacaaggcgaacctttcgcggccattctcgacgacgtcaataacctggtcatgcccggcctgtcccattggcagcacccggacttctatggctatttcccttccaatggcaccctgtcctcggtgctgggggacttcctcagtaccggtctgggcgtgctgggcctgtcctggcaatccagcccggccctgagcgaactggaagaaaccaccctcgactggctgcgccagttgcttggcctgtctggccagtggagtggggtgatccaggacactgcctcgaccagcaccctggtggcgctgatcagtgcccgtgaacgcgccactgactacgccctggtacgtggtggcctgcaggccgagcccaagcctttgatcgtgtatgtcagcgcccacgcccacagctcggtggacaaggctgcactgctggcaggttttggccgcgacaatatccgcctgattcccaccgacgaacgctacgccctgcgcccagaggcactgcaggcggcgatcgaacaggacctggctgccggcaaccagccgtgcgccgtggttgccaccaccggcaccacgacgaccactgccctcgacccgctgcgcccggtcggtgaaatcgcccaggccaatgggctgtggttgcacgttgactcggccatggccggttcggcgatgatcctgcccgagtgccgctggatgtgggacggcatcgagctggccgattcggtggtggtcaacgcgcacaaatggctgggtgtggccttcgattgctcgatctactacgtgcgcgatccgcaacacctgatccgggtgatgagcaccaatcccagctacctgcagtcggcggtggatggcgaggtgaagaacctgcgcgactgggggataccgctgggccgtcggttccgtgcgttgaagctgtggttcatgttgcgcagcgagggtgtcgacgcattgcaggcgcggctgcggcgtgacctggacaatgcccagtggctggcggggcaggtcgaggcggcggcggagtgggaagtgttggcgccagtacagctgcaaaccttgtgcattcgccatcgaccggcggggcttgaaggggaggcgctggatgcgcataccaagggctgggccgagcggctgaatgcatccggcgctgcttatgtgacgccggctacactggacgggcggtggatggtgcgggtttcgattggtgcgctgccgaccgagcggggggatgtgcagcggctgtgggcacgtctgcaggacgtgatcaagggctga

SEQ ID NO: 10 - protein, 470 aa, Pseudomonas putida
MTPEQFRQYGHQLIDLIADYRQTVGERPVMAQVEPGYLKAALPATAPQQGEPFAAILDDVNNLVMPGLSHWQHPDFYGYFPSNGTLSSVLGDFLSTGLGVLGLSWQSSPALSELEETTLDWLRQLLGLSGQWSGVIQDTASTSTLVALISARERATDYALVRGGLQAEPKPLIVYVSAHAHSSVDKAALLAGFGRDNIRLIPTDERYALRPEALQAAIEQDLAAGNQPCAVVATTGTTTTTALDPLRPVGEIAQANGLWLHVDSAMAGSAMILPECRWMWDGIELADSVVVNAHKWLGVAFDCSIYYVRDPQHLIRVMSTNPSYLQSAVDGEVKNLRDWGIPLGRRFRALKLWFMLRSEGVDALQARLRRDLDNAQWLAGQVEAAAEWEVLAPVQLQTLCIRHRPAGLEGEALDAHTKGWAERLNASGAAYVTPATLDGRWMVRVSIGALPTERGDVQRLWARLQDVIKG

SEQ ID NO: 11 - DNA, 2274 bp, Escherichia coli
atgggaagcccctctctgtattctgcccgtaaaacaaccctggcgttggcagtcgccttaagtttcgcctggcaagcgccggtatttgcccacggtggtgaagcgcatatggtgccaatggataaaacgcttaaagaatttggtgccgatgtgcagtgggacgactacgcccagctctttaccctgattaaagatggcgcgtacgtgaaagtgaagcctggtgcgcaaacagcaattgttaatggtcagcctctggcactgcaagtaccggtagtgatgaaagacaataaagcctgggtttctgacacctttattaacgatgttttccagtccgggctggatcaaacctttcaggtagaaaagcgccctcacccacttaatgcgctaactgcggacgaaattaaacaggccgttgaaattgttaaagcttccgcggacttcaaacccaatacccgttttactgagatctccctgctaccgccagataaagaagctgtctgggcgtttgcgctggaaaacaaaccggttgaccagccgcgcaaagccgacgtcattatgctcgacggcaaacatatcatcgaagcggtggtggatctgcaaaacaacaaactgctctcctggcaacccattaaagacgcccacggtatggtgttgctggatgatttcgccagtgtgcagaacattattaacaacagtgaagaatttgccgctgccgtgaagaaacgcggtattactgatgcgaaaaaagtgattaccacgccgctgaccgtaggttatttcgatggtaaagatggcctgaaacaagatgcccggttgctcaaagtcatcagctatcttgatgtcggtgatggcaactactgggcacatcccatcgaaaacctggtggcggtcgttgatttagaacagaaaaaaatcgttaagattgaagaaggtccggtagttccggtgccaatgaccgcacgcccatttgatggccgtgaccgcgttgctccggcagttaagcctatgcaaatcattgagcctgaaggtaaaaattacaccattactggcgatatgattcactggcggaactgggattttcacctcagcatgaactctcgcgtcgggccgatgatctccaccgtgacttataacgacaatggcaccaaacgcaaagtcatgtacgaaggttctctcggcggcatgattgtgccttacggtgatcctgatattggctggtactttaaagcgtatctggactctggtgactacggtatgggcacgctaacctcaccaattgctcgtggtaaagatgccccgtctaacgcagtgctccttaatgaaaccatcgccgactacactggcgtgccgatggagatccctcgcgctatcgcggtatttgaacgttatgccgggccggagtataagcatcaggaaatgggccagcccaacgtcagtaccgaacgccgggagttagtggtgcgctggatcagtacagtgggtaactatgactacatttttgactggatcttccatgaaaacggcactattggcatcgatgccggtgctacgggcatcgaagcggtgaaaggtgttaaagcgaaaaccatgcacgatgagacggcgaaagatgacacgcgctacggcacgcttatcgatcacaatatcgtgggtactacacaccaacatatttataatttccgcctcgatctggatgtagatggcgagaataacagcctggtggcgatggacccagtggtaaaaccgaatactgccggtggcccacgcaccagtaccatgcaagttaatcagtacaacatcggcaatgaacaggatgccgcacagaaatttgatccgggcacgattcgtctgttgagtaacccgaacaaagagaaccgcatgggcaatccggtttcctatcaaattattccttatgcaggtggtactcacccggtagcaaaaggtgcccagttcgcgccggacgagtggatctatcatcgtttaagctttatggacaagcagctctgggtaacgcgttatcatcctggcgagcgtttcccggaaggcaaatatccgaaccgttctactcatgacaccggtcttggacaatacagtaaggataacgagtcgctggacaacaccgacgccgttgtctggatgaccaccggcaccacacatgtggcccgcgccgaagagtggccgattatgccgaccgaatgggtacatactctgctgaaaccatggaacttctttgacgaaacgccaacgctaggggcgctgaagaaagataagtga

SEQ ID NO: 12 - protein, 757 aa, Escherichia coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK

SEQ ID NO: 13 - DNA, 1191 bp, Artificial Sequence
  synthetic gene based on the gene sequence from Methanocaldococcus jannaschii using the codon bias of E. coli (tyrD)
atgcgcaacatgcaggaaaaaggcgtgtctgaaaaagaaatcctggaagaactgaagaaataccgttccctggatctgaagtatgaagacggtaacatttttggtagcatgtgctccaatgtactgccgattacccgcaaaattgtcgatatttttctggagactaacctgggtgatccaggcctgtttaagggcaccaaactgctggaagaaaaggccgtagctctgctgggctctctgctgaacaacaaagacgcatacggtcacattgtgtctggtggcaccgaagccaacctgatggcgctgcgttgcattaaaaacatctggcgtgaaaaacgtcgcaagggtctgtccaaaaacgagcacccgaaaattatcgttccaattactgctcacttctcctttgaaaaaggtcgcgaaatgatggacctggaatatatctacgctcctatcaaagaagattacactatcgacgagaagttcgtgaaggatgctgtggaagactacgacgtggacggtattatcggcatcgcgggtactaccgaactgggtacgatcgacaacattgaggagctgtctaaaatcgcgaaggaaaacaatatctacatccacgtggacgcagcgttcggtggtctggttatcccatttctggatgacaaatacaaaaagaagggtgttaactacaaattcgacttcagcctgggcgtagacagcattaccatcgatcctcacaagatgggccattgcccaattccgagcggcggtatcctgttcaaagacatcggttacaaacgttacctggacgtggacgctccgtacctgactgaaactcgtcaggcgacgatcctgggcactcgtgtgggctttggcggtgcgtgtacctatgctgtgctgcgttatctgggtcgtgagggtcagcgtaagatcgtgaacgaatgcatggaaaacaccctgtacctgtacaaaaagctgaaagaaaacaacttcaaaccggttatcgagccgatcctgaacattgtggccatcgaagacgaagattacaaagaagtttgtaagaagctgcgtgatcgcggtatctacgtgtctgtgtgtaactgcgttaaggccctgcgtatcgtggtaatgccgcacatcaaacgcgaacacatcgataacttcatcgagattctgaactctatcaaacgcgattaa

SEQ ID NO: 14 - protein, 396 aa, Methanocaldococcus jannaschii
MRNMQEKGVSEKEILEELKKYRSLDLKYEDGNIFGSMCSNVLPITRKIVDIFLETNLGDPGLFKGTKLLEEKAVALLGSLLNNKDAYGHIVSGGTEANLMALRCIKNIWREKRRKGLSKNEHPKIIVPITAHFSFEKGREMMDLEYIYAPIKEDYTIDEKFVKDAVEDYDVDGIIGIAGTTELGTIDNIEELSKIAKENNIYIHVDAAFGGLVIPFLDDKYKKKGVNYKFDFSLGVDSITIDPHKMGHCPIPSGGILFKDIGYKRYLDVDAPYLTETRQATILGTRVGFGGACTYAVLRYLGREGQRKIVNECMENTLYLYKKLKENNFKPVIEPILNIVAIEDEDYKEVCKKLRDRGIYVSVCNCVKALRIVVMPHIKREHIDNFIEILNSIKRD

SEQ ID NO: 15 - DNA, 3908 bp, Acinetobacter calcoaceticus
gatcatcagtgcagcaaccaccaacatcatcaagaaaccaaatgcttcacttggcatcatgtaattgatcaccacaaccaatgcggtcactgctgatgagatcagcacagcattcattggaataccacgcgtattcactttggttaagaatttgggtgcattgccctgttctgccaaaccatgcagcattcgtgtattacaatatacacagctgttatagaccgacactgccgcaatcaacaccacaaagttcaatacgttggcaacgccattactatcaagcgaatggaaaatcatgacaaatggactaccgccttctgcaacttgattccaaggatataaactgagtaggatcgtaatcgcaccgatatagaaaattaaaacacgataaacaatttgattggtggctttcggaattgatttcttcggatctttggtttcagccgcgctaatcccaattaactctaacccaccaaaggcaaacatgattgcagccatcgccatcataaacccttgtgcaccattcgggaaaaagccaccgagttgccataggttcgatacactcgcttggggacctgctgttccgctaaatagcaaataagcaccaaaagcaatcatactcaaaatggcaaaaatcttaatcaaggaaaggacaaactccgtttcaccaaagaaacgtacgttgatcaagttaatcccgttaattaagacaaagaaaaataatgcagatgcccacgtcggtaactctcgccaccaaaattgcatgaaggttccaatggcactcagttccgccatgcccaccaacacataaagcacccagtaattccagccagacatgaagcctgccattctgccccaatacttatgtgcaaaatggctaaatgaaccactcacaggctcttcaacgaccatttcaccaagttggcgcataattaaaaatgcaatgacacctgccaaggcataaccgagaatcactgaaggaccagccaatttaatggtttgtgaaagccctagaaataatcctgtgccaattgcaccacccagtgcaatcagctggatatgacgattcgtcaaatccttctttaaaccgttagatttctcaatttccataattttccctaacttgctataaattccattacagcattttatcattcatataaacagaactttaaagccttgttcctgtttttatctcgcttgcatgtgcttcctttactcaggttagttatgcttaaaagattattcatcactacagcacaaaaccgacagcatccatttcatctatacaaatcaattatttatttgattctacttaggatggagtttgacttatacgttttgcattgaatcatgactcaacacaaaagatcgtcatgtatgcgcgatcagcttattttcaaccctatgcaaatctagctaaccaagttttctataccgatttaatttccaaacatcctctgtttggcacacttgaatcctaactgctgacgcttaaaaatacaatataattccatgtatttctacatcttaattaaaaacaaatacacttcgaattgaagaaagaattgtaatttacttctcaatgctaatctaaattaagtgtttttaatgcattatttgggccgataatcacacgacttcatcccagtgatggcagataaaataagactgacgatttcgatatctcaaaattacggttcaagaaaaaaacaaaaaaactttttacttgtaatagatgatatctaaatcgcgcgtcttaaatcatgttgtttactaaacaaccaactagaatgcaatcgcctttttatattcggtatcttgctgaggatgtttcagtttttgataggtcatatcaccataatcataacgtgcaaccaaatctgcaaatttggttaaattacttggtgcatccattgcagctagctttaactcgaccacggcaagatttggatgctcagaaagttgcgctaaagtgtctttgagctcagctaaattttcgaccataaatgtatcgtattgaccttgaccattaaagaccttgaccatctcggtatatttccagttttgaacatcgttatatttcgcgttctcacctaaaatcagacgttcaatggtatagccgccattgtttaaaataaaaataatcggctttaggtcttcgcgaataatagttgacaactcttgcatggtcagctgaatcgaaccatccccaataaacagcacatgacgtcgtttgggtgcagcgaccatactgccgagcaatgctggtaaggtataaccaattgatccccaaagtggttgtgagatatagcgggcttgtttcggtaaacgcatactcgataacgcagaatttgacgtgcctacctcaccaataatcacatcatcatcacgcaagaactgccccacttcttgccacaattgcaagtgtgtcaatggacgttttaactcttcttctgcaggtacaggagtactcgcggccagtggtgccgctaaagtcggtttagagactttacgcacagcaacctgatcaagcaaattgcttaacagctcttgaatttcaatcccagggtagttttcttgatcgatcgtgacatcgtattgtttaatctcaatataatgatctgtattaatacgatgagtaaagtaagcggaaccaacatcactaaaacgcacaccaatcccaatcaagcaatctgactgttcaatcaactttctggttgctgcaggccccacagcaccgacatatacgccggcatataatggagaggattcatccatggtgtttttggtggtatttaaacacgcataaggaatgccacatttttctgcaagttgtcccagcaatgtcgtcacttggaaggtatgtgcatcatgatcaatcagtaatgcaggattcttggcttggctaatctgttcgctgagtaactgcacaacatgtgcaagcacctctggatcacttttcggtttagacaaatctagtgtacgaccatcgacgtcgattttgacatgcgtaatatcagaaggaagttggatatagactggacgacgttcaatccaacattgacgcaatacccggtcaatttcagcagcagcatttgcaggagtaatacgcgtttgagcaacactgaactctttcatacaatttaaaatattttgataattgccatcaaccaaggtgtgatgcagtaatgcgccctgttctacagcgtgtaatggcggtataccagagatcaccacaacaggtactttttctgcatacgcgcccgcaacgccattgatagcactgagatcacctacaccataggtggtgagtaaagcaccaaaaccattgatacgcgcataaccatctgcggcataggctgcatttaattcattacaattgccaataaatgccaatttagcatctgcttcaacttgctctaaataacttaaattaaagtcacctggcacaccaaaaagatgctgtacaccaagctcagccagacgctgatttaaataacaaccaatctcaataaacatttctacttccctgcaaaataattgttgttataaacagaataggtcaattcattttgtatattcgtgcataatagagtcataaatttaaaaaaatgcacagaatgtgtatcgcaaaggaatttcatgcaatggataagtttgattggcaaatcattcatgcgttacaacgcaacggtaggctcaccaatcaagaaattggcgatttgattggcctttctgcctctcaatgttcccgcagaagacaagttcttgaacaaaaaagtattattttaggctatagcgcaagaataaatccaaatgcgcttggaatttcaattaccaccatgattcatgtcaacctcaagaaccacggcgccaatcccaaacatgccatacatgatctgatc

SEQ ID NO: 16 - DNA, 1731 bp, Acinetobacter calcoaceticus
atgtttattgagattggttgttatttaaatcagcgtctggctgagcttggtgtacagcatctttttggtgtgccaggtgactttaatttaagttatttagagcaagttgaagcagatgctaaattggcatttattggcaattgtaatgaattaaatgcagcctatgccgcagatggttatgcgcgtatcaatggttttggtgctttactcaccacctatggtgtaggtgatctcagtgctatcaatggcgttgcgggcgcgtatgcagaaaaagtacctgttgtggtgatctctggtataccgccattacacgctgtagaacagggcgcattactgcatcacaccttggttgatggcaattatcaaaatattttaaattgtatgaaagagttcagtgttgctcaaacgcgtattactcctgcaaatgctgctgctgaaattgaccgggtattgcgtcaatgttggattgaacgtcgtccagtctatatccaacttccttctgatattacgcatgtcaaaatcgacgtcgatggtcgtacactagatttgtctaaaccgaaaagtgatccagaggtgcttgcacatgttgtgcagttactcagcgaacagattagccaagccaagaatcctgcattactgattgatcatgatgcacataccttccaagtgacgacattgctgggacaacttgcagaaaaatgtggcattccttatgcgtgtttaaataccaccaaaaacaccatggatgaatcctctccattatatgccggcgtatatgtcggtgctgtggggcctgcagcaaccagaaagttgattgaacagtcagattgcttgattgggattggtgtgcgttttagtgatgttggttccgcttactttactcatcgtattaatacagatcattatattgagattaaacaatacgatgtcacgatcgatcaagaaaactaccctgggattgaaattcaagagctgttaagcaatttgcttgatcaggttgctgtgcgtaaagtctctaaaccgactttagcggcaccactggccgcgagtactcctgtacctgcagaagaagagttaaaacgtccattgacacacttgcaattgtggcaagaagtggggcagttcttgcgtgatgatgatgtgattattggtgaggtaggcacgtcaaattctgcgttatcgagtatgcgtttaccgaaacaagcccgctatatctcacaaccactttggggatcaattggttataccttaccagcattgctcggcagtatggtcgctgcacccaaacgacgtcatgtgctgtttattggggatggttcgattcagctgaccatgcaagagttgtcaactattattcgcgaagacctaaagccgattatttttattttaaacaatggcggctataccattgaacgtctgattttaggtgagaacgcgaaatataacgatgttcaaaactggaaatataccgagatggtcaaggtctttaatggtcaaggtcaatacgatacatttatggtcgaaaatttagctgagctcaaagacactttagcgcaactttctgagcatccaaatcttgccgtggtcgagttaaagctagctgcaatggatgcaccaagtaatttaaccaaatttgcagatttggttgcacgttatgattatggtgatatgacctatcaaaaactgaaacatcctcagcaagataccgaatataaaaaggcgattgcattc

SEQ ID NO: 17 - protein, 577 aa, Acinetobacter calcoaceticus
MFIEIGCYLNQRLAELGVQHLFGVPGDFNLSYLEQVEADAKLAFIGNCNELNAAYAADGYARINGFGALLTTYGVGDLSAINGVAGAYAEKVPVVVISGIPPLHAVEQGALLHHTLVDGNYQNILNCMKEFSVAQTRITPANAAAEIDRVLRQCWIERRPVYIQLPSDITHVKIDVDGRTLDLSKPKSDPEVLAHVVQLLSEQISQAKNPALLIDHDAHTFQVTTLLGQLAEKCGIPYACLNTTKNTMDESSPLYAGVYVGAVGPAATRKLIEQSDCLIGIGVRFSDVGSAYFTHRINTDHYIEIKQYDVTIDQENYPGIEIQELLSNLLDQVAVRKVSKPTLAAPLAASTPVPAEEELKRPLTHLQLWQEVGQFLRDDDVIIGEVGTSNSALSSMRLPKQARYISQPLWGSIGYTLPALLGSMVAAPKRRHVLFIGDGSIQLTMQELSTIIREDLKPIIFILNNGGYTIERLILGENAKYNDVQNWKYTEMVKVFNGQGQYDTFMVENLAELKDTLAQLSEHPNLAVVELKLAAMDAPSNLTKFADLVARYDYGDMTYQKLKHPQQDTEYKKAIAF

SEQ ID NO: 18 - DNA, 1506 bp, Ralstonia pickettii
atggcgttactggaacgcgccgcgtggtacgacatcgcacgcacgaccaactggaccccgagctacgtcaccgagtccgagctgtttcccgacatcatgaccggcgcgcagggcgtaccgatggagacctgggaaacctacgacgaaccctacaagacgtcgtatcccgaatacgtcagcattcaacgcgagaaggatgccggagcgtactcggtcaaggccgcgctggagcgcagccgcatgttcgaagacgccgacccgggctggctgtcgatcctgaaggcgcactacggcgccattgcgctcggcgaatacgcagcgatgagcgccgaggcacgcatggcccgcttcggccgcgcgccgggcatgcgcaacatggccaccttcggcatgctcgatgagaaccggcacggccagctgcagttgtatttcccgcacgactattgcgccaaggaccgtcagttcgattgggcccataaggcttatcacaccaacgaatggggcgcgatcgcggcacgcagcacgttcgacgatctgttcatgtcgcgcagcgcgatcgacattgcgatcatgctcacgttcgcgttcgagacgggctttaccaacatgcagttcctcggtctcgcggccgacgctgcagaggcgggggatttcacctttgccagcctgatctcaagcatccagaccgacgagtcgcggcatgcacagatcggtggtccggctctgcagatcctgatcgcaagcggccgcaaggaacaggcgcagaaactcgtcgacatcgccattgcgcgggcctggcggctgttctcgctgctcaccggcacctcgatggattacgcaacgccgctgcaccatcgcaaggagtcgttcaaggagttcatgactgagtggatcgtcgggcagtttgaacgcaccttgatcgacctgggcctggacctgccctggtactgggatcagatgatcaacgagttcgactaccagcatcacgcctatcagatgggcatctggttctggcgcccgacgatctggtggaaccccgctgccggcatcacgcccgattgccgcgactggctcgaagagaaataccccggctggaacgacacgttcggcaaggcctgggacgtcatcatcgacaacctgctggccggcaagcccgagctgaccgtgcccgagacactgcccatcgtctgcaacatgagccagttgccgatctgcgcggttccgggtaacggctggatcgtgaaggactacccgctcgactacaagggccgcacgtaccacttcaattccgagatcgaccgctgggtcttccagcaggacccgctgcgctatcgcgaccacctgacgctggtcgaccgattcctcgccggccagatccagccgcccaacctgatgggcgcgcttcagtacatgaacctggcgcctggcgagtgcggcgacgacgcccatcactacgcgtgggtcgaggcgtaccgcaatcagcgctaccagaagaaagccgcttga

SEQ ID NO: 19 - protein, 501 aa, Ralstonia pickettii
MALLERAAWYDIARTTNWTPSYVTESELFPDIMTGAQGVPMETWETYDEPYKTSYPEYVSIQREKDAGAYSVKAALERSRMFEDADPGWLSILKAHYGAIALGEYAAMSAEARMARFGRAPGMRNMATFGMLDENRHGQLQLYFPHDYCAKDRQFDWAHKAYHTNEWGAIAARSTFDDLFMSRSAIDIAIMLTFAFETGFTNMQFLGLAADAAEAGDFTFASLISSIQTDESRHAQIGGPALQILIASGRKEQAQKLVDIAIARAWRLFSLLTGTSMDYATPLHHRKESFKEFMTEWIVGQFERTLIDLGLDLPWYWDQMINEFDYQHHAYQMGIWFWRPTIWWNPAAGITPDCRDWLEEKYPGWNDTFGKAWDVIIDNLLAGKPELTVPETLPIVCNMSQLPICAVPGNGWIVKDYPLDYKGRTYHFNSEIDRWVFQQDPLRYRDHLTLVDRFLAGQIQPPNLMGALQYMNLAPGECGDDAHHYAWVEAYRNQRYQKKAA

SEQ ID NO: 20 - DNA, 990 bp, Ralstonia pickettii
atgacaacgcaagctgaagtcctcaagccgctcaagacctggagccatctggccgcgcggcgacgcaagcccagcgagtacgaaatcgtctcgaccaacctgcactacaccaccgacaacccggatgcgccgttcgaactcgacccgaatttcgagatggcgcagtggttcaagcgcaaccgcaacgcatcgcccctgacccaccccgactggaacgcgttccgcgatccggatgaactggtctaccgcacgtacaacatgctgcaggacgggcaggagacctatgtgttcgggctgctcgaccagttttccgagcgcgggcacgacgccatgctcgaacgcacctgggccggcacgctggcacgcctgtacacgcccgtgcgctacctgttccacacgctgcagatgggctcggcctatctgacgcaactggcgcccgcctcgaccatctcgaactgcgcggcgtaccagacggccgattcgctgcgctggctgacacacaccgcttaccgcaccaaggagctgtcgcagaccttcagcgacctcggcttcggcaccgatgaacgccgctactgggagcaggacccggcctggcaaggctggcgcaagctggtcgaacacgcgctggtggcgtgggactgggccgagtgcttcgttgccctgagcctggtggtgcggccggcagtggaggaagccgtcttgcgcagcctcggcgaagccgcccggcataacggcgacaccttgctgggcctgctgaccgacgcgcaactcgccgatgcgcaacgccatcggcgctgggccggcgcattggtgcgcatggcgctggagcaacccggaaaccgcgaagtcatcaccggttggctcgccaagtgggagcccctggcggatgaagccatcgtggcctactgctcggccctgcccgaggcgcctgcggcccaggcacgcgcaaccgctgcggtgcgcgagttccggcacagcctcggcctgtga

SEQ ID NO: 21 - protein, 329 aa, Ralstonia pickettii
MTTQAEVLKPLKTWSHLAARRRKPSEYEIVSTNLHYTTDNPDAPFELDPNFEMAQWFKRNRNASPLTHPDWNAFRDPDELVYRTYNMLQDGQETYVFGLLDQFSERGHDAMLERTWAGTLARLYTPVRYLFHTLQMGSAYLTQLAPASTISNCAAYQTADSLRWLTHTAYRTKELSQTFSDLGFGTDERRYWEQDPAWQGWRKLVEHALVAWDWAECFVALSLVVRPAVEEAVLRSLGEAARHNGDTLLGLLTDAQLADAQRHRRWAGALVRMALEQPGNREVITGWLAKWEPLADEAIVAYCSALPEAPAAQARATAAVREFRHSLGL

SEQ ID NO: 22 - DNA, 261 bp, Ralstonia pickettii
atggcactttttcctgtgatttccaactttcagtacgacttcgtgctgcaactcgtcgcggtggatacggaaaacaccatcgacgaggtggccgcagcagcggcacaccactcggtgggacgccgcgtggcaccgcagcccggcaagatcgtcagggtgcggcgccagggcggcgagcagttctacccgcgtaacgccaggctggccgacaccgacatcaagccgatggaagcgctcgaattcattttttgcgatgcatga

SEQ ID NO: 23 - protein, 86 aa, Ralstonia pickettii
MALFPVISNFQYDFVLQLVAVDTENTIDEVAAAAAHHSVGRRVAPQPGKIVRVRRQGGEQFYPRNARLADTDIKPMEALEFIFCDA

SEQ ID NO: 24 - DNA, 1503 bp, Pseudomonas mendocina KR1
atggcgatgcacccacgtaaagactggtatgaactgaccagggcgacaaattggacacctagctatgttaccgaagagcagcttttcccagagcggatgtccggtcatatgggtatcccgctggaaaaatgggaaagctatgatgagccctataagacatcctatccggagtacgtaagtatccaacgtgaaaaggatgcaggtgcttattcggtgaaggcggcacttgagcgtgcaaaaatttatgagaactctgacccaggttggatcagcactttgaaatcccattacggcgccatcgcagttggtgaatatgcagccgtaaccggtgaaggtcgtatggcccgtttttcaaaagcaccgggaaatcgcaacatggctacgtttggcatgatggatgaactgcgccatggccagttacagctgtttttcccgcatgaatactgtaagaaggatcgccagtttgattgggcatggcgggcctatcacagtaacgaatgggcagccattgctgcaaagcatttctttgatgacatcattaccggacgtgatgcgatcagcgttgcgatcatgttgacgttttcattcgaaaccggcttcaccaacatgcagtttcttgggttggcggcagatgccgcagaagcaggtgactacacgtttgcaaacctgatctccagcattcaaaccgatgagtcgcgtcatgcacaacagggcggccccgcattacagttgctgatcgaaaacggaaaaagagaagaagcccaaaagaaagtcgacatggcaatttggcgtgcctggcgtctatttgcggtactaaccgggccggttatggattactacacgccgttggaggaccgcagccagtcattcaaggagtttatgtacgagtggatcatcggacagttcgaacgctcgttgatagatctgggcttggacaagccctggtactgggatctattcctcaaggatattgatgagcttcaccatagttatcacatgggtgtttggtactggcgtacaaccgcttggtggaaccctgctgccggggtcactcctgaggagcgtgactggctggaagaaaagtatccaggatggaataaacgttggggtcgttgctgggatgtgatcaccgaaaacgttctcaatgaccgtatggatcttgtctctccagaaaccttgcccagcgtgtgcaacatgagccagataccgctggtaggtgttcctggtgatgactggaatatcgaagttttcagtcttgagcacaatgggcgtctttatcattttggctctgaagtggatcgctgggtattccagcaagatccggttcagtatcaaaatcatatgaatatcgtcgaccgcttcctcgcaggtcagatacagccgatgactttggaaggtgccctcaaatatatgggcttccaatctattgaagagatgggcaaagacgcccacgactttgcatgggctgacaagtgcaagcctgctatgaagaaatcggcctga

SEQ ID NO: 25 - protein, 500 aa, Pseudomonas mendocina KR1
MAMHPRKDWYELTRATNWTPSYVTEEQLFPERMSGHMGIPLEKWESYDEPYKTSYPEYVSIQREKDAGAYSVKAALERAKIYENSDPGWISTLKSHYGAIAVGEYAAVTGEGRMARFSKAPGNRNMATFGMMDELRHGQLQLFFPHEYCKKDRQFDWAWRAYHSNEWAAIAAKHFFDDIITGRDAISVAIMLTFSFETGFTNMQFLGLAADAAEAGDYTFANLISSIQTDESRHAQQGGPALQLLIENGKREEAQKKVDMAIWRAWRLFAVLTGPVMDYYTPLEDRSQSFKEFMYEWIIGQFERSLIDLGLDKPWYWDLFLKDIDELHHSYHMGVWYWRTTAWWNPAAGVTPEERDWLEEKYPGWNKRWGRCWDVITENVLNDRMDLVSPETLPSVCNMSQIPLVGVPGDDWNIEVFSLEHNGRLYHFGSEVDRWVFQQDPVQYQNHMNIVDRFLAGQIQPMTLEGALKYMGFQSIEEMGKDAHDFAWADKCKPAMKKSA

SEQ ID NO: 26 - DNA, 984 bp, Pseudomonas mendocina KR1
atgagctttgaatccaagaaaccgatgcgtacatggagccacctggccgaaatgagaaagaagccaagtgagtacgatattgtctcacgcaagcttcactacagtaccaacaatcccgattcaccctgggagctgagccccgatagcccaatgaatctgtggtacaagcagtaccgtaacgcatcgccattgaaacacgataactgggatgcttttactgatcctgaccaacttgtataccgcacctacaacctgatgcaggatggtcaggaatcttatgtgcagagtctgttcgatcaattcaatgagcgcgaacatgaccaaatggtgcgggagggctgggagcacacaatggcccgctgttattccccgttgcgctatctgttccactgcctgcagatgtcgtcggcctatgttcagcagatggcgccggcgagcacaatctcaaattgctgcatccttcaaactgctgacagcctgcgatggttgacgcacaccgcctaccgaacgcacgaactcagtcttacttatccggatgctggtttaggtgagcacgagcgagaactgtgggagaaagagccgggttggcaggggctgcgtgaattgatggagaagcaactaactgcttttgattggggagaggcttttgtcagtctaaatttggtggtcaagccaatgattgtcgagagtattttcaaaccactgcagcagcaagcatgggaaaataacgataccttgcttcctctgttgattgacagtcagctgaaagatgccgagcgtcatagtcgttggtcgaaagcacttgtaaaacatgcgctggaaaaccccgataatcacgctgtaattgaaggttggattgaaaagtggcgccccttggctgacagggcagctgaagcttacctgagtatgctatctagcgacattttgcccgctcaatatcttgagcgtagtacctcattgagggcatccatacttacggtctga

SEQ ID NO: 27 - protein, 327 aa, Pseudomonas mendocina KR1
MSFESKKPMRTWSHLAEMRKKPSEYDIVSRKLHYSTNNPDSPWELSPDSPMNLWYKQYRNASPLKHDNWDAFTDPDQLVYRTYNLMQDGQESYVQSLFDQFNEREHDQMVREGWEHTMARCYSPLRYLFHCLQMSSAYVQQMAPASTISNCCILQTADSLRWLTHTAYRTHELSLTYPDAGLGEHERELWEKEPGWQGLRELMEKQLTAFDWGEAFVSLNLVVKPMIVESIFKPLQQQAWENNDTLLPLLIDSQLKDAERHSRWSKALVKHALENPDNHAVIEGWIEKWRPLADRAAEAYLSMLSSDILPAQYLERSTSLRASILTV

SEQ ID NO: 28 - DNA, 255 bp, Pseudomonas mendocina KR1
atgtcggcatttccagttcacgcagcgtttgaaaaagatttcttggttcaactggtagtggtggatttaaatgattccatggaccaggtagcggagaaagttgcctaccattgtgttaatcgtcgtgttgctcctcgtgaaggtgtcatgcgggttcgaaagcatagatcaactgagctatttccacgggatatgaccatagctgagagcggccttaacccaactgaagtgatcgatgtggtattcgaggagtag

SEQ ID NO: 29 - protein, 84 aa, Pseudomonas mendocina KR1
MSAFPVHAAFEKDFLVQLVVVDLNDSMDQVAEKVAYHCVNRRVAPREGVMRVRKHRSTELFPRDMTIAESGLNPTEVIDVVFEE

SEQ ID NO: 30 - DNA, 789 bp, Pseudomonas aeruginosa
atgaaaacgacgcagtacgtggcccgccagcccgacgacaacggtttcatccactatccggaaaccgagcaccaggtctggaataccctgatcacccggcaactgaaggtgatcgaaggccgcgcctgtcaggaatacctcgacggcatcgaacagctcggcctgccccacgagcggatcccccagctcgacgagatcaacagggttctccaggccaccaccggctggcgcgtggcacgggttccggcgctgattccgttccagaccttcttcgaactgctggccagccagcaattccccgtcgccaccttcatccgcaccccggaagaactggactacctgcaggagccggacatcttccacgagatcttcggccactgcccactgctgaccaacccctggctcgccgagttcacccatacctacggcaagctcggcctcaaggcgagcaaggaggaacgcgtgttcctcgcccgcctgtactggatgaccatcgagttcggcctggtcgagaccgaccagggcaagcgcatctacggcggcggcatcctctcctcgccgaaggagaccgtctacagcctctccgacgagccgctgcaccaggccttcaatccgctggaggcgatgcgcacgccctaccgcatcgacatcctgcaaccgctctatttcgtcctgcccgacctcaagcgcctgttccaactggcccaggaagacatcatggcgctggtccacgaggccatgcgcctgggcctgcacgcgccgctgttcccgcccaagcaggcggcctga

SEQ ID NO: 31 - protein, 262 aa, Pseudomonas aeruginosa
MKTTQYVARQPDDNGFIHYPETEHQVWNTLITRQLKVIEGRACQEYLDGIEQLGLPHERIPQLDEINRVLQATTGWRVARVPALIPFQTFFELLASQQFPVATFIRTPEELDYLQEPDIFHEIFGHCPLLTNPWLAEFTHTYGKLGLKASKEERVFLARLYWMTIEFGLVETDQGKRIYGGGILSSPKETVYSLSDEPLHQAFNPLEAMRTPYRIDILQPLYFVLPDLKRLFQLAQEDIMALVHEAMRLGLHAPLFPPKQAA

SEQ ID NO: 32 - DNA, 357 bp, Pseudomonas aeruginosa
atgaccgcactcacccaagcccattgcgaagcctgccgcgcagacgccccgcacgtcagcgacgaagaactgcccgtgctgctgcggcaaatcccggattggaacatcgaagtccgcgacggcatcatgcagctagagaaggtctacctgttcaagaacttcaagcatgccctggccttcaccaatgccgtcggcgagatatccgaggccgaaggccaccatccgggcctgctgaccgagtggggcaaagtcaccgtgacctggtggagccactcgatcaagggcctgcaccgcaacgatttcatcatggcggcgcgcaccgatgaggtagcgaaaaccgccgaggggcgcaaatga

SEQ ID NO: 33 - protein, 118 aa, Pseudomonas aeruginosa
MTALTQAHCEACRADAPHVSDEELPVLLRQIPDWNIEVRDGIMQLEKVYLFKNFKHALAFTNAVGEISEAEGHHPGLLTEWGKVTVTWWSHSIKGLHRNDFIMAARTDEVAKTAEGRK

SEQ ID NO: 34 - DNA, 789 bp, Pseudomonas putida
atgaaacagacgcaatacgtggcacgcgagcccgatgcgcatggttttatcgattacccgcagcaagagcatgcggtgtggaacaccctgatcacccgccagctgaaagtgatcgaaggccgtgcgtgccaggaatacctggacggcatcgaccagctgaaattgccgcatgaccgcattccgcaactgggcgagatcaacaaggtgctgggtgccaccaccggctggcaggttgcccgggttccggcgctgatccccttccagaccttcttcgaattgctggccagcaagcgctttccggtcgccaccttcatccgcaccccggaagagctggactacctgcaagagccggatatcttccacgagatcttcggccactgcccgctgctgaccaatccctggttcgccgaattcacccacacctacggcaagctcggcctggccgcgaccaaggaacaacgtgtgtacctggcacgcttgtactggatgaccatcgagtttggcctgatggaaaccgcgcaaggccgcaaaatctatggtggtggcatcctctcgtcgccgaaagagaccgtctacagtctgtctgacgagcctgagcaccaggccttcgacccgatcgaggccatgcgtacaccctaccgcatcgacattctgcaaccggtgtatttcgtactgccgaacatgaagcgcctgttcgacctggcccacgaggacatcatgggcatggtccataaagccatgcagctgggtctgcatgcaccgaagtttccacccaaggtcgctgcctga

SEQ ID NO: 35 - protein, 262 aa, Pseudomonas putida
MKQTQYVAREPDAHGFIDYPQQEHAVWNTLITRQLKVIEGRACQEYLDGIDQLKLPHDRIPQLGEINKVLGATTGWQVARVPALIPFQTFFELLASKRFPVATFIRTPEELDYLQEPDIFHEIFGHCPLLTNPWFAEFTHTYGKLGLAATKEQRVYLARLYWMTIEFGLMETAQGRKIYGGGILSSPKETVYSLSDEPEHQAFDPIEAMRTPYRIDILQPVYFVLPNMKRLFDLAHEDIMGMVHKAMQLGLHAPKFPPKVAA

SEQ ID NO: 36 - DNA, 357 bp, Pseudomonas putida
atgaatgccttgaaccaagcccattgcgaagcctgccgcgccgacgcaccgaaagtctccgacgaagagctggccgagctgattcgcgaaatcccggactggaacattgaagtacgtgacggccacatggagcttgagcgcgtgttcctgttcaagaacttcaagcacgccttggcgttcaccaacgccgtgggcgaaatcgccgaagccgaaggccaccacccagggctgctgaccgagtggggcaaggttaccgtcacttggtggagccactcgatcaaaggcctgcaccgcaacgacttcatcatgtgcgcgcgcactgacaaggtggctgaatcggctgaaggccgtaagtaa

SEQ ID NO: 37 - protein, 118 aa, Pseudomonas putida
MNALNQAHCEACRADAPKVSDEELAELIREIPDWNIEVRDGHMELERVFLFKNFKHALAFTNAVGEIAEAEGHHPGLLTEWGKVTVTWWSHSIKGLHRNDFIMCARTDKVAESAEGRK

SEQ ID NO: 38 - DNA, 1707 bp, Agaricus bisporus
atgtctcatctgctcgtttctcctcttggaggaggcgttcaacctcgtcttgaaataaataattttgtaaagaatgaccgtcaattctctctttacgttcaagctctcgaccggatgtacgccacccctcagaatgaaactgcgtcctactttcaagtagctggagtgcatggatacccactcatccctttcgatgatgcagtcggtccaaccgagttcagtccttttgaccaatggactgggtattgcactcacggctcaactctttttccaacttggcatcgtccttatgttttgattctcgaacaaattttgagtggacacgctcaacaaatcgccgatacttacactgtcaataaatccgagtggaaaaaggcggcaaccgaattccgtcatccgtattgggattgggcatctaatagcgttcctcctccggaagtcatctccctacccaaagtcactatcacgactccgaatggccaaaagacgagcgtcgccaacccactgatgaggtatactttcaactctgtcaacgacggcggtttctatgggccgtataatcagtgggatactactttgagacaacccgactcgacgggtgtgaacgcaaaggataacgttaataggcttaaaagtgttttgaaaaatgctcaagccagtcttacacgggctacttacgacatgttcaaccgcgtcacgacttggcctcatttcagcagccatactcctgcgtctggaggaagtaccagtaatagtatcgaggcaattcatgacaatatccatgtcctcgtcggtggtaacggccacatgagtgatccttctgtcgccccctttgatcctatcttcttcttgcatcatgcgaacgttgatcgactgattgctttatggtcggctattcgttacgatgtgtggacttccccgggcgacgctcaatttggtacatatactttgagatataagcagagtgttgacgagtcgaccgaccttgctccgtggtggaagactcaaaatgaatactggaaatccaatgaactgaggagcaccgagtcgttgggatacacttaccccgagtttgttggtttggatatgtacaacaaagacgcggtaaacaagaccatttcccgaaaggtagcacagctttatggaccacaaagaggagggcaaaggtcgctcgtagaggatttatcaaactcccatgctcgtcgtagtcaacgccctgcgaagcgctcccgccttggtcaactcttgaaagggttattctcggattggtctgctcaaatcaaattcaaccgccatgaagtcggccagagcttctcggtttgtcttttcctgggcaatgttcctgaagacccgagggagtggttggttagccccaacttggttggcgctcgtcatgcgttcgtccgttcggtcaagaccgaccatgtagccgaggaaataggtttcattccgattaaccagtggattgccgagcacacgggtttaccttcgtttgcagtagaccttgtaaaaccactcttggcacaaggtttacagtggcgcgtgctcttggcggatggaacccctgctgagctcgattcactggaagtgactatattggaggtcccatccgagctgaccgacgatgagcctaatccccgctccaggccgcccaggtaccacaaggatattacacacggaaagcgtggtggttgccgcgaggcttga

SEQ ID NO: 39 - protein, 568 aa, Agaricus bisporus
MSHLLVSPLGGGVQPRLEINNFVKNDRQFSLYVQALDRMYATPQNETASYFQVAGVHGYPLIPFDDAVGPTEFSPFDQWTGYCTHGSTLFPTWHRPYVLILEQILSGHAQQIADTYTVNKSEWKKAATEFRHPYWDWASNSVPPPEVISLPKVTITTPNGQKTSVANPLMRYTFNSVNDGGFYGPYNQWDTTLRQPDSTGVNAKDNVNRLKSVLKNAQASLTRATYDMFNRVTTWPHFSSHTPASGGSTSNSIEAIHDNIHVLVGGNGHMSDPSVAPFDPIFFLHHANVDRLIALWSAIRYDVWTSPGDAQFGTYTLRYKQSVDESTDLAPWWKTQNEYWKSNELRSTESLGYTYPEFVGLDMYNKDAVNKTISRKVAQLYGPQRGGQRSLVEDLSNSHARRSQRPAKRSRLGQLLKGLFSDWSAQIKFNRHEVGQSFSVCLFLGNVPEDPREWLVSPNLVGARHAFVRSVKTDHVAEEIGFIPINQWIAEHTGLPSFAVDLVKPLLAQGLQWRVLLADGTPAELDSLEVTILEVPSELTDDEPNPRSRPPRYHKDITHGKRGGCREA

SEQ ID NO: 40 - DNA, 1671 bp, Agaricus bisporus
atgtcgctgattgctactgtcggacctactggcggagtcaagaaccgtctgaacatcgttgattttgtgaagaatgaaaagtttttcacgctttatgtacgctccctcgaacttctacaagccaaggaacagcatgactactcgtctttcttccaactagccggcattcatggtctaccctttactgagtgggccaaagagcgaccttccatgaacctatacaaggctggttattgtacccatgggcaggttctgttcccgacttggcatagaacgtacctttctgtgttggagcaaatacttcaaggagctgccatcgaagttgctaagaagttcacttctaatcaaaccgattgggtccaggcggcgcaggatttacgccagccctactgggattggggtttcgaacttatgcctcctgatgaggttatcaagaacgaagaggtcaacattacgaactacgatggaaagaagatttccgtcaagaaccctatcctccgctatcacttccatccgatcgatccttctttcaagccatacggggactttgcaacctggcgaacaacagtccgaaaccccgatcgtaataggcgagaggatatccctggtctaatcaaaaaaatgagacttgaggaaggtcagattcgtgagaagacctacaatatgttgaagttcaacgatgcttgggagagattcagtaaccacggcatatctgatgatcagcatgctaacagcttggagtctgttcacgatgacattcatgttatggttggatacggcaaaatcgaaggacatatggaccaccctttctttgctgccttcgacccgattttctggttacatcataccaacgtcgaccgtctactatccctttggaaagcaatcaaccccgatgtgtgggttacgtcgggacgtaaccgggatggtaccatgggcatcgcacccaacgctcagatcaacagcgagacccctcttgagccattctaccaatctggggataaagtgtggacctcggcctctctcgctgatactgctcggctcggctactcctaccccgatttcgacaagttggttggaggaacaaaggagttgattcgcgacgctatcgacgacctcatcgatgagcggtatggaagcaaaccttcgagtggggctcgcaatactgcctttgatctcctcgccgatttcaagggcattaccaaagagcacaaggaggatctcaaaatgtacgactggaccatccatgttgccttcaagaagttcgagttgaaagagagtttcagtcttctcttctactttgcgagtgatggtggcgattatgatcaggagaattgctttgttggatcaattaacgccttccgtgggactgctcccgaaacttgcgcgaactgccaagataacgagaacttgattcaagaaggctttattcacttgaatcattatcttgctcgtgaccttgaatctttcgagccgcaggacgtgcacaagttcttaaaggaaaaaggactgtcatacaaactctacagcaggggagataaacctttgacatcgttgtcagttaagattgaaggacgtccccttcatctaccgcccggagagcatcgtccgaagtacgatcacactcaggcccgagtagtgtttgatgatgtcgcggtgcatgttattaactga

SEQ ID NO: 41 - protein, 556 aa, Agaricus bisporus
MSLIATVGPTGGVKNRLNIVDFVKNEKFFTLYVRSLELLQAKEQHDYSSFFQLAGIHGLPFTEWAKERPSMNLYKAGYCTHGQVLFPTWHRTYLSVLEQILQGAAIEVAKKFTSNQTDWVQAAQDLRQPYWDWGFELMPPDEVIKNEEVNITNYDGKKISVKNPILRYHFHPIDPSFKPYGDFATWRTTVRNPDRNRREDIPGLIKKMRLEEGQIREKTYNMLKFNDAWERFSNHGISDDQHANSLESVHDDIHVMVGYGKIEGHMDHPFFAAFDPIFWLHHTNVDRLLSLWKAINPDVWVTSGRNRDGTMGIAPNAQINSETPLEPFYQSGDKVWTSASLADTARLGYSYPDFDKLVGGTKELIRDAIDDLIDERYGSKPSSGARNTAFDLLADFKGITKEHKEDLKMYDWTIHVAFKKFELKESFSLLFYFASDGGDYDQENCFVGSINAFRGTAPETCANCQDNENLIQEGFIHLNHYLARDLESFEPQDVHKFLKEKGLSYKLYSRGDKPLTSLSVKIEGRPLHLPPGEHRPKYDHTQARVVFDDVAVHVIN